SEQUENCE LISTING (31 sequences)

SEQ ID NO: 1 - DNA, 627 bp, Artificial
  dsWsv477
atgtatatcttcgtcgaaggttcccccctcacagggaagagttcatggatgtccaagttgatagatacaggatcatgtggaatgtctttcctcaattttcttcgtatgaacacttctgactactacaactggcctgccgaaatcgggacagaacatctccagttaggtttcagagaaaccagagtggtggatggaatgtttgaacctgtcctaaagacctttgtcgactcgtggaagaaagagcaaggaaaagagagtttgaaggaatatctggactacaacggccaagtcatggagatctacatcgcagaatggttgagacaaaggccactagccttccacgtgtttacctatacagatgaagctgtcaagagtggattcttgaacgaggaggatctagatatggatactgcaaccaagtggatggctgaaattattagagagaagaggggcaatattcaagaaataaaagtgacccctagagtagtcttcaatggcaatggttgtagtgcatgtttctctaacactaagagaaacttgtataactttggaacaaactataacaatgttgtacattgtgatttgttgtgcccttttgcaaggcataggattgtacatttcttataa

SEQ ID NO: 2 - DNA, 601 bp, Artificial
  dsVp28
tcactctttcggtcgtgtcggccatcctcgccatcactgctgtgattgctgtatttattgtgatttttaggtatcacaacactgtgaccaagaccatcgaaacccacacaggcaatatcgagacaaacatggatgaaaacctccgcattcctgtgactgctgaggttggatcaggctacttcaagatgactgatgtgtcctttgacagcgacaccttgggcaaaatcaagatccgcaatggaaagtctgatgcacagatgaaggaagaagatgcggatcttgtatcactcccgtggagggccgagcactcgaagtgactgtggggcggaatctcacctttgaggggacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagtaa

SEQ ID NO: 3 - DNA, 350 bp, Artificial
  dsVp19
gccaccacgactaacactcttcctttcggcaggaccggagcccaggccgctggcccttcttacaccatggaagatcttgaaggctccatgtctatggctcgcatgggtctctttttgatcgttgctatctcaattggtatcctcgtcctggccgtcatgaatgtatggatgggaccaaagaaggacagcgattctgacactgataaggacaccgatgatgatgacgacactgccaacgataacgatgatgaggacaaatataagaacaggaccagggatatgatgcttctggctgggtccgctcttctgttcctcgtttccgccgccaccgtttttatgtcttaccccaa

SEQ ID NO: 4 - DNA, 681 bp, E. coli
atgaaccccatcgtaattaatcggcttcaacggaagctgggctacacttttaatcatcaggaactgttgcagcaggcattaactcatcgtagtgccagcagtaaacacaatgcccgcttggaatttttaggcgactctattctgagctacgttatcgccaatgcgctttatcaccgtttccctcgtgtggatgaaggcgatatgagccggatgcgcgccacgctggtccgtggcaatacgctggcggaactggcgcgcgaatttgagttaggcgagtgcttacgtttagggccaggtgaacttaaaagcggtggatttgccgccgagtcaattctcgccgacaccgtcgaagcattaattggtggcgtattcctcgacagtgatattcaaaccgtcgagaaattaatcctcaactggtatcaaactcgtttggacgaaattagcccaggcgataaacaaaaagatccgaaaacgcgcttgcaagaatatttgcagggtcgccatctgccgctgccgacttatctggtagtccaggtacgtggcgaagcgcacgatcaggaatttactatccactgccaggtcagcggcctgagtgaaccggtggttggcacaggttcaagccgtcgtaaggctgagcaggctgccgccgaacaggcgttgaaaaaactggagctggaatga

SEQ ID NO: 5 - protein, 226 aa, E. coli
MNPIVINRLQRKLGYTFNHQELLQQALTHRSASSKHNARLEFLGDSILSYVIANALYHRFPRVDEGDMSRMRATLVRGNTLAELAREFELGECLRLGPGELKSGGFAAESILADTVEALIGGVFLDSDIQTVEKLILNWYQTRLDEISPGDKQKDPKTRLQEYLQGRHLPLPTYLVVQVRGEAHDQEFTIHCQVSGLSEPVVGTGSSRRKAEQAAAEQALKKLELE

SEQ ID NO: 6 - DNA, 681 bp, Enterobacteriaceae Ag001
atgaatcccatcgtaataaataggctgcagcgtaagctgggctacacttttcaacatcaggatctgttgcaacaggcattaacccatcggagtgccagcagcaagcataatgcccgcttggagtttttgggtgactccattctcagttatgtcatcgcgaatgcgctgtatcatcgttttcctcgcgtagatgaaggcgacatgagccgcatgcgtgcgacgctggtgcgcggcaatacgctggcggaaatcgcccgcgagttcgaactgggtgagtgtctgcgtcttgggccgggtgaactgaaaagtggcggtttccgtcgcgagtcgattcttgctgataccgtggaagcgttgatcggtggcgtcttcctcgacagcgacattcagaacgttgagcgtttgattctctcgtggtatcagacccgtctcgacgaaatcagtccaggcgacaagcaaaaagatccgaaaacgcgtctgcaggagtacctgcagggtcgccatctgccgctgccgtcgtatctggtggtgcaggtgcgtggtgaagcgcacgatcaagaatttaccattcactgtcaggtgagtggcctgcctgagcctgtcgtagggacgggctcaagccgccgtaaagcggaacaggctgcggctgagcaggcactgaaaaagctggagctggaatga

SEQ ID NO: 7 - protein, 226 aa, Enterobacteriaceae Ag001
MNPIVINRLQRKLGYTFQHQDLLQQALTHRSASSKHNARLEFLGDSILSYVIANALYHRFPRVDEGDMSRMRATLVRGNTLAEIAREFELGECLRLGPGELKSGGFRRESILADTVEALIGGVFLDSDIQNVERLILSWYQTRLDEISPGDKQKDPKTRLQEYLQGRHLPLPSYLVVQVRGEAHDQEFTIHCQVSGLPEPVVGTGSSRRKAEQAAAEQALKKLELE

SEQ ID NO: 8 - DNA, 681 bp, Enterobacteriaceae Ag001
atgaatcccatcgtaataaataggctgcagcgtaagctgggctacacttttcaacatcaggatctgttgcaacaggcattaacccatcggagtgccagcagcaagcataatgcccgcttggagtttttgggtgactccattctcagttatgtcatcgcgaatgcgctgtatcatcgttttcctcgcgtagatgaaggcgacatgagccgcatgcgtgcgacgctggtgcgcggcaatacgctggcggaaatcgcccgcgagttcgaactgggtgagtgtctgcgtcttgggccgggtgaactgaaaagtggcggtttcgccgccgagtcgattcttgctgataccgtggaagcgttgatcggtggcgtcttcctcgacagcgacattcagaacgttgagcgtttgattctctcgtggtatcagacccgtctcgacgaaatcagtccaggcgacaagcaaaaagatccgaaaacgcgtctgcaggagtacctgcagggtcgccatctgccgctgccgtcgtatctggtggtgcaggtgcgtggtgaagcgcacgatcaagaatttaccattcactgtcaggtgagtggcctgcctgagcctgtcgtagggacgggctcaagccgccgtaaagcggaacaggctgcggctgagcaggcactgaaaaagctggagctggaatga

SEQ ID NO: 9 - protein, 226 aa, Enterobacteriaceae Ag001
MNPIVINRLQRKLGYTFQHQDLLQQALTHRSASSKHNARLEFLGDSILSYVIANALYHRFPRVDEGDMSRMRATLVRGNTLAEIAREFELGECLRLGPGELKSGGFAAESILADTVEALIGGVFLDSDIQNVERLILSWYQTRLDEISPGDKQKDPKTRLQEYLQGRHLPLPSYLVVQVRGEAHDQEFTIHCQVSGLPEPVVGTGSSRRKAEQAAAEQALKKLELE

SEQ ID NO: 10 - DNA, 681 bp, Enterobacter Ae003
atgaaccccatcgtaattaatcggcttcaacggaagctgggctacacttttcatcatcaggagttgttgcaacaggcattaacccaccgcagtgccagcagcaagcacaacgcccgcctggagtttttaggcgactctattttaagtttcgtgattgcgaatgcgctttatcatcgtttcccgcgcgtggatgaaggtgatatgagccgcatgcgtgccacgctggttcggggtaacacccttgcggaaatcgcgcgcgaatttgaactgggcgaatgtctgcgtcttgggccgggtgaactgaaaagcggcggcttccgtcgtgaatctattcttgccgatacggtcgaagcattaattggtggtgtgttcctggacagcgatatccagaccgtcgaaaagctgatcctgaactggtatcagacccgtctggacgaaatcagcccgggcgataaacaaaaagatcccaaaacgcgtctgcaggaatatttgcagggccgtcatctgccgctgccatcttatctggtggtgcaggttcgtggcgaagcgcacgatcaggaatttaccatccattgccaggtcagtggcctgagtgaaccggtggtgggcacaggttcaagccgtcgtaaggctgaacaggctgccgccgaacaggcgttaaaaatgctggagctggaatga

SEQ ID NO: 11 - protein, 226 aa, Enterobacter Ae003
MNPIVINRLQRKLGYTFHHQELLQQALTHRSASSKHNARLEFLGDSILSFVIANALYHRFPRVDEGDMSRMRATLVRGNTLAEIAREFELGECLRLGPGELKSGGFRRESILADTVEALIGGVFLDSDIQTVEKLILNWYQTRLDEISPGDKQKDPKTRLQEYLQGRHLPLPSYLVVQVRGEAHDQEFTIHCQVSGLSEPVVGTGSSRRKAEQAAAEQALKMLELE

SEQ ID NO: 12 - DNA, 681 bp, Enterobacter Ae003
atgaaccccatcgtaattaatcggcttcaacggaagctgggctacacttttcatcatcaggagttgttgcaacaggcattaacccaccgcagtgccagcagcaagcacaacgcccgcctggagtttttaggcgactctattttaagtttcgtgattgcgaatgcgctttatcatcgtttcccgcgcgtggatgaaggtgatatgagccgcatgcgtgccacgctggttcggggtaacacccttgcggaaatcgcgcgcgaatttgaactgggcgaatgtctgcgtcttgggccgggtgaactgaaaagcggcggcttcgccgccgaatctattcttgccgatacggtcgaagcattaattggtggtgtgttcctggacagcgatatccagaccgtcgaaaagctgatcctgaactggtatcagacccgtctggacgaaatcagcccgggcgataaacaaaaagatcccaaaacgcgtctgcaggaatatttgcagggccgtcatctgccgctgccatcttatctggtggtgcaggttcgtggcgaagcgcacgatcaggaatttaccatccattgccaggtcagtggcctgagtgaaccggtggtgggcacaggttcaagccgtcgtaaggctgaacaggctgccgccgaacaggcgttaaaaatgctggagctggaatga

SEQ ID NO: 13 - protein, 226 aa, Enterobacter Ae003
MNPIVINRLQRKLGYTFHHQELLQQALTHRSASSKHNARLEFLGDSILSFVIANALYHRFPRVDEGDMSRMRATLVRGNTLAEIAREFELGECLRLGPGELKSGGFAAESILADTVEALIGGVFLDSDIQTVEKLILNWYQTRLDEISPGDKQKDPKTRLQEYLQGRHLPLPSYLVVQVRGEAHDQEFTIHCQVSGLSEPVVGTGSSRRKAEQAAAEQALKMLELE

SEQ ID NO: 14 - protein, 121 aa, White spot syndrome virus
MATTTNTLPFGRTGAQAAGPSYTMEDLEGSMSMARMGLFLIVAISIGILVLAVMNVWMGPKKDSDSDTDKDTDDDDDTANDNDDEDKYKNRTRDMMLLAGSALLFLVSAATVFMSYPKRRQ

SEQ ID NO: 15 - DNA, 366 bp, White spot syndrome virus
atggccaccacgactaacactcttcctttcggcaggaccggagcccaggccgctggcccttcttacaccatggaagatcttgaaggctccatgtctatggctcgcatgggtctctttttgatcgttgctatctcaattggtatcctcgtcctggccgtcatgaatgtatggatgggaccaaagaaggacagcgattctgacactgataaggacaccgatgatgatgacgacactgccaacgataacgatgatgaggacaaatataagaacaggaccagggatatgatgcttctggctgggtccgctcttctgttcctcgtttccgccgccaccgtttttatgtcttaccccaagaggaggcagtaa

SEQ ID NO: 16 - protein, 172 aa, White spot syndrome virus
VTKTIETHTDNIETNMDENLRIPVTAEVGSGYFKMTDVSFDSDTLGKIKIRNGKSDAQMKEEDADLVITPVEGRALEVTVGQNLTFEGTFKVWNNTSRKINITGMQMVPKINPSKAFVGSSNTSSFTPVSIDEDEVGTFVCGTTFGAPIAATAGGNLFDMYVHVTYSGTETE

SEQ ID NO: 17 - DNA, 516 bp, White spot syndrome virus
gtgaccaagaccatcgaaacccacacagacaatatcgagacaaacatggatgaaaacctccgcattcctgtgactgctgaggttggatcaggctacttcaagatgactgatgtgtcctttgacagcgacaccttgggcaaaatcaagatccgcaatggaaagtctgatgcacagatgaaggaagaagatgcggatcttgtcatcactcccgtggagggccgagcactcgaagtgactgtggggcagaatctcacctttgagggaacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgag

SEQ ID NO: 18 - protein, 208 aa, White spot syndrome virus
MYIFVEGSPLTGKSSWMSKLIDTGSCGMSFLNFLRMNTSDYYNWPAEIGTEHLQLGFRETRVVDGMFEPVLKTFVDSWKKEQGKESLKEYLDYNGQVMEIYIAEWLRQRPLAFHVFTYTDEAVKSGFLNEEDLDMDTATKWMAEIIREKRGNIQEIKVTPRVVFNGNGCSACFSNTKRNLYNFGTNYNNVVHCDLLCPFARHRIVHFL

SEQ ID NO: 19 - DNA, 627 bp, White spot syndrome virus; identical to SEQ ID NO: 1

SEQ ID NO: 20 - DNA, 7409 bp, E. coli
atgcaaacgagtgatacccgcgcgttaccgctactttgcgcccgctcggtttataaacagtattcaggggtcaatgtcctgaaaggcatcgattttacgttgcatcagggggaggtccacgccctgctcggcggcaatggtgccggtaaatcgacgttaatgaagattattgccggtattacccctgctgatagcggtacgctggagattgagggcaacaactacgtcagattaacgccagttcatgctcatcagctgggtatttatctcgttccccaggaaccgctgcttttcccaagcctgtcgataaaagaaaacatcctgtttgggctggcaaaaaaacagctctccatgcagaaaatgaagaacttgctggcggcgctgggctgccagtttgatctgcatagtctggcaggatcgctggatgtcgccgatcgccaaatggtggaaatcctccgcgggctgatgcgcgactcgcggattctgatcctcgatgaacctaccgcctcgcttacccctgcggaaaccgaacgcttgtttagtcgcttgcaagagctgcttgctactggcgtgggtattgtttttatctcgcataagctgccggaaattcgccagattgccgatcgaattagcgtgatgcgcgacggaaccatcgccttaagcggcaaaaccagcgaactgtctaccgacgacattattcaggccatcaccccagcggtacgggaaaaatcgctctctgccagccaaaaattatggctggagttacctggtaaccgcccacaacatgccgccggaacgccggtgctgacactggaaaatctgaccggcgaaggtttcaggaatgtcagcctgacgctcaatgccggagaaattctgggcctggctgggctggtgggggccggacgcacagaactggccgagacgctctatggtctgcgtactttgcgtggcggacgcattatgctgaatggtaaagagatcaataaattatccactggagaacgtttactgcgcggtctggtttatctgccggaagatcgccagtcatccggactgaatctcgatgcttcgctggcctggaacgtctgcgcccttactcataaccttcgtggattctgggcgaaaaccgcgaaagataatgccaccctggaacgttatcgtcgggcgctgaatattaaattcaaccaaccggaacaagctgcacggacattatccggtggcaaccagcaaaaaatcctcattgccaaatgcttggaagcttcgccgcaagtattgattgtcgatgagccgacgcgcggcgtggatgtctcggcccgtaatgatatctaccagctgttgcgcagcatcgccgcacaaaatgtggctgtgctgcttatctcctccgacctggaagagatcgaactgatggcagatcgtgtgtatgtgatgcatcagggcgaaattacccactctgcactgaccgagcgcgatattaatgtcgagactattatgcgcgttgccttcggcgatagtcagcgtcaggaggcgtcatgctgaagtttattcagaacaaccgtgaaatcacggcactgctggcggtggtgctgctgtttgtattacccggttttctcgaccgccagtatttaagtgtgcaaacgctgaccatggtttatagcagcgcgcaaatcctgatcctgctggcaatgggcgcgacgctggtaatgcttacgcgcaatattgatgtttcagtgggttcgattaccggaatgtgcgcggtgctgttggggatgttactgaacgcaggatattcactacctgttgcttgtgtcgcgactttactgcttggtttgctcgcgggatttttcaacggtgtcctggtcgcgtggctaaagatccctgccattgttgccacccttggcacgttagggttgtacagaggcatcatgttgctgtggactggcggcaaatggattgaagggttacccgccgaactgaaacagctctccgccccgctgctgcttggcgtttcagcaattggttggttgacgataattctggtggcatttatggcctggctgctggcaaagacggcgtttggacgcagtttttatgccacgggcgataatttacagggcgctcgtcaactgggcgttcgtactgaagccattcgcattgtggcattttcgttgaacggctgcatggcggcactggcgggaattgtgtttgcttcgcagattggttttatccccaaccagaccggtaccgggctggagatgaaagcaattgcagcctgcgtgctgggcggcattagtttgctcggtggttccggtgcgatcattggtgcggtactcggcgcatggttcctgacgcagatcgatagcgtactggtgctgttgcgcattccggcatggtggaatgattttatcgcgggtctggttctgctggcggtgctggtgtttgatggacgcctgcgttgtgcgctggaacgtaatctacggcggcaaaaatatgcccgctttatgacgccaccgccatccgttaaacccgcttcgtcaggtaaaaaacgggaggccgcataatgcgtattcgctacggttgggaactggctcttgccgcactgctcgttattgagattgtcgcatttggtgcaattaacccgcgaatgttagatctcaatatgttgctgttcagcaccagtgactttatctgcattggcattgtcgccctaccgctaacgatggtgattgtcagtggcgggatcgatatttcgtttggttcgaccatcggcctctgcgccattgcattgggcgtactgtttcaaagtggtgtgccgatgccgctggcgatactcctgaccttactgctcggcgcattgtgcgggctgatcaacgccggattaattatctataccaaagttaacccgctggtgattacgcttggcacgctgtatctgtttgccggaagcgctctgctgctttccggtatggccggagcgacggggtacgaaggtattggtggattcccgatggcgtttacagatttcgctaacctggatgtgctgggactccccgttccgctgattatcttcctgatatgtctcctcgttttctggctctggctgcataaaacccatgccggacgtaatgtgtttttgattgggcaaagcccgcgcgtggcgctttatagcgcgattccagttaaccgtaccttatgtgcgctctatgccatgacggggctggcgtctgcggtcgccgctgtgctgctggtatcgtattttggttcagcacgttccgatctcggtgcgtcgtttctgatgcccgccatcaccgccgtggtgcttggcggggccaatatttatggtggttccggttccattatcggcaccgccattgcggttttattagtgggatatttgcaacaaggtttgcaaatggcaggagtgccaaatcaggtgtccagcgccctttccggtgcgctacttatcgtcgttgtcgtaggtcgttccgttagcctgcatcgccagcaaattaaagagtggctggcgcgtcgggccaataacccattgccataaaggatatcttcatgacacttcatcgctttaagaaaatcgccttacttagcgctcttggcattgccgcaatctctatgaatgtgcaggccgcagagcgtattgcatttattcccaaactggttggcgtgggattttttaccagcggtggcaacggcgcacaacaagcgggtaaagagctgggcgttgatgtgacctacgacgggccgacagaacccagtgtttctggtcaggtacagttgattaataacttcgtcaatcaaggttataacgccattatcgtttctgcggtttcgcctgatggcttgtgtccggcactgaaacgcgccatgcaacgtggtgtgagagtgctgacctgggactctgatactaaaccggagtgccgctcttactacattaatcagggaacgcccgcccagttaggaggtatgttggtggatatggcggcgcgtcaggtgaataaagacaaagccaaagtcgcgtttttctactcaagccccaccgttacggaccaaaaccagtgggtgaaagaagcgaaagcgaaaatcgccaaagagcatccaggctgggaaattgtcactacgcagtttggctataacgatgccactaaatcgttacaaaccgcagaaggaatattaaaagcgtatagcgatctcgacgccattatcgcccccgatgccaacgccctgcccgctgccgcacaagccgcagaaaacttgaaaaatgacaaagtagcgattgtcggattcagtacgccaaatgtgatgcgcccgtatgtagagcgcggcacggtgaaagaatttggcctgtgggatgtggttcagcaaggcaaaatttcagtgtatgtcgcggatgcattattgaaaaaaggatcaatgaaaacgggcgacaagctggatatcaagggcgtaggtcaggttgaagtctcgccaaacagcgttcagggctatgactacgaagcggatggtaatggcatcgtactgttaccggagcgcgtgatattcaacaaagagaatatcggcaaatacgatttctgatgtgcattacttaaccggagtaagttatggcagatttagacgatattaaagatggtaaagattttcgtaccgatcaaccgcaaaaaaatatcccttttaccctgaaaggttgcggtgcgctggattggggaatgcagtcacgcttatcgcggatatttaatccgaaaacgggtaaaaccgtgatgctggcttttgaccatggttattttcagggaccgactaccggacttgaacgcattgatataaatatcgccccgctgtttgaacatgccgatgtattaatgtgtacgcgcggcattttgcgcagcgtagttccccctgcgaccaataggccggtggtactgcgggcgtcaggtgcgaactctattctggcggaattaagtaatgaagccgtggcgttatcgatggatgacgccgtgcgcctgaacagttgcgcggtggcggcgcaggtttatatcggcagcgaatatgaacatcagtcgatcaaaaatattattcagctggttgatgccggaatgaaagtgggaatgccgaccatggccgtgactggcgtgggcaaagatatggtgcgcgatcagcgttatttctcgctcgcgactcgaatcgccgctgaaatgggggcgcaaattatcaaaacctattatgtcgaaaaaggttttgaacggattgttgccggatgtccggtacccattgttattgctggcggtaaaaaattaccggagcgcgaggcgctggaaatgtgctggcaggctatcgatcagggcgcttctggtgtggatatggggcgtaatattttccagtctgaccatccggtggcgatgatgaaagccgtacaggcggtggttcaccataacgaaacggctgatcgggcatatgaactctatctgagtgaaaaacagtaactgcggatctaaggagaagaattatgcacgtcacactggttgaaattaacgttcatgaagacaaggttgacgagtttatcgaagtttttcgccagaaccacctgggctctgtacaggaagaaggcaatttgcgcttcgatgtcttacaggacccggaagtgaattcgcgcttttatatctacgaagcctataaagatgaagacgcagtggcgttccataaaaccacgccccactacaaaacctgtgtcgcgaaactggaatctttaatgaccgggccgcgtaaaaaacgtctgttcaatggtttgatgccgtgaatctacctcgaggtttatggctcgactctttaccctttcagaatcaaagtactacctgatggcgctggatgcaggcaccggaagtattcgggctgtgatattcgacctggaaggcaatcaaatagcagtgggacaggcggagtggcggcatctggcagtaccggacgttcctggttctatggaatttgatctcaacaaaaactggcaactggcgtgtgagtgtatgcgccaggcgctgcacaacgccggcatagccccggagtatatcgctgccgtttcggcatgttcgatgcgtgaaggcattgttttatataataatgaaggagccccgatctgggcctgcgccaatgtggatgccagagcggcacgcgaagttagcgaacttaaagaactgcacaacaatacctttgaaaacgaagtttatcgcgcgaccggacaaacactggctttaagtgccatccccagattactttggctggcgcaccatcgttccgatatttaccgtcaggcatcaaccatcaccatgatcagcgactggctggcctatatgctcagcggcgaactggcggtggatccctctaacgctggcaccacgggacttcttgatctaaccacccgtgactggaaacctgcattgctggatatggctggcctacgtgccgatattctttctcctgtcaaagaaaccggcacattgctgggcgtggtaagttcacaagcggcggaactctgcggtctgaaggcgggcactccggtggtcgttggaggaggcgacgtgcagcttggttgccttgggttaggcgttgtgcgtccggcacaaaccgcggttcttggcggcacattctggcagcaagttgtaaatttagccgcgccggtgacagacccagaaatgaacgtgcgcgttaatcctcatgttattcctggcatggtacaagctgaatctataagcttttttaccggactcaccatgcgctggttccgcgatgctttctgtgccgaagaaaaactgattgccgaacgtttaggcatcgacacctatacgctgctggaagagatggccagtcgggtgccgcctgggtcgtggggcgtaatgccgatcttctccgacagaatgcgctttaaaacctggtatcacgctgcgccttcctttattaacttgtccattgacccggataaatgtaacaaagcgacattgttccgtgcgctggaagaaaatgcggcgattgtatcagcgtgtaacttgcagcaaattgctgatttctcgaatattcatccttcatcgttagtctttgcaggcggaggttcaaaagggaaattatggagtcaaattctcgctgatgtctcgggattacccgtcaatattccggtggtcaaagaagccactgcattaggatgtgccattgcagctggcgtcggtgccggaattttttcatcaatggcagaaaccggagaacgcctggttcgctgggaacggacgcacacaccagacccggaaaagcatgaactttatcaggattcacgcgataagtggcaggcagtttatcaggatcagctggggctggttgatcatggactgacgacgtcgttatggaaagcgcctgggttatag

SEQ ID NO: 21 - DNA, 816 bp, Ochrobactrum
atgacaattaattatcatgaattggaaaccagtcacggtcgtatcgcagtccgtgagtcagaaggggaaggtgcgccgctgctgatgatacatggtaatagcagttccggggcgatttttgcgccacagcttgagggggaaataggaaagaaatggcgtgtcattgccccagatctgccgggacatggaaagagcacggacgcaatcgacccggaccgctcttacagcatggaaggctacgctgatgccatgacagaggttatgcaacaactcggtattgcagatgcggtggtattcggctggagccttggaggtcatattggcatagaaatgatcgcgcgttacccagaaatgcgtggtttaatgattacgggcacccctccggttgcacgggaagaagtaggacaaggctttaagagtggtccagatatggcgcttgcaggtcaagaaattttttcagaacgggatgttgagtcttacgctcggagtacgtgcggagaaccttttgaagctagtcttttggacatcgtagcacggactgacgggcgggctagacgcattatgttcgaaaaatttgggagtggaactggcggtaaccaacgggacatcgttgctgaagcacaattacctattgccgtagtgaatgggcgggatgaaccatttgtcgagttggacttcgttagcaaagttaaatttggaaacctctgggaaggtaaaactcatgtaatcgacaatgcgggacatgctcctttccgggaagctccagctgagttcgatgcatatctcgcccgcttcatacgtgattgtacgcagtaa

SEQ ID NO: 22 - RNA, 54 nt, Artificial
  anti-sense RNA sequence targeting expression of dam gene in Vibrio
uaauucaacaagaauugggacaacuccagugaaaaguucuucuccuuuacucau

SEQ ID NO: 23 - DNA, 840 bp, Vibrio sp.
atgaaaaagcaacgagcctttcttaagtgggcaggaggcaaatacggtctggttgaagacatccaacgtcatttaccaccggctcgaaagctagttgaaccctttgttggtgctggctcggtttttctaaataccgactatgaccactatctactggcggatattaaccccgacctgattaatctctataacttactaaaagagcgtcctgaagagtacatctcagaagcgaagcgctggtttgttgcagagaacaatcgcaaagaagcgtacttgaatattcgcgccgagtttaataaaacggatgacgtgatgtaccgctcgttggcgttcctatacatgaaccgctttggctttaatggcttatgtcgttataacaaaaaaggcggctttaatgtcccgtttggttcttacaaaaagccttatttcccagaagcggagctagaattctttgctgaaaaagccaagaaagcgacgttcgtatgtgaaggttacccagaaacgttcagtcgagcgcgtaaaggcagcgtggtttattgcgatccaccgtacgcaccgttgtcgaacacggcgaactttacctcttatgctggcaacggctttacgctggatgatcaagctgcattggctgatattgcagagaaagccgcaactgaacgtggtatccctgttctgatctcaaaccatgacacgacattaacgcgtcgcctttatcatggtgcggagcttaatgtcgtaaaagtgaagcgaaccatcagtcgtaatggcagtggtcgtaataaagttgacgagttgctggcgctatttcgtgcacctgacgcggacaaatctgactcttaa

SEQ ID NO: 24 - protein, 409 aa, Vibrio parahaemolyticus
MTNEYVVTMSSLTEFNPNNARKSYLFDNYEVDPNYAFKAMVSFGLSNIPYAGGFLSTLWNIFWPNTPNEPDIENIWEQLRDRIQDLVDESIIDAINGILDSKIKETRDKIQDINETIENFGYAAAKDDYIGLVTHYLIGLEENFKRELDGDEWLGYAILPLLATTVSLQITYMACGLDYKDEFGFTDSDVHKLTRNIDKLYDDVSSYITELAAWADNDSYNNANQDNVYDEVMGARSWCTVHGFEHMLIWQKIKELKKVDVFVHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKPSVRLQLEGHFICGM

SEQ ID NO: 25 - protein, 408 aa, Vibrio parahaemolyticus
MTNEYVVTMSSLTEFNPNNARKSYLFDNYEVDPNYAFKAMVSFGLSNIPYAGGFLSTLWNIFWPNTPNEPDIENIWEQLRDRIQDLVDESIIDAINGILDSKIKETRDKIQDINETIENFGYAAAKDDYIGLVTHYLIGLEENFKRELDGDEWLGYAILPLLATTVSLQITYMACGLDYKDEFGFTDSDVHKLTRNIDKLYDDVSSYITELAAWADNDSYNNANQDNVYDEVMGARSWCTVHGFEHMLIWQKIKELKKVDVFVHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKPSVRLQLEGHFICG

SEQ ID NO: 26 - protein, 385 aa, Vibrio parahaemolyticus
MTNEYVVTMSSLTEFNPNNARKSYLFDNYEVDPNYAFKAMVSFGLSNIPYAGGFLSTLWNIFWPNTPNEPDIENIWEQLRDRIQDLVDESIIDAINGILDSKIKETRDKIQDINETIENFGYAAAKDDYIGLVTHYLIGLEENFKRELDGDEWLGYAILPLLATTVSLQITYMACGLDYKDEFGFTDSDVHKLTRNIDKLYDDVSSYITELAAWADNDSYNNANQDNVYDEVMGARSWCTVHGFEHMLIWQKIKELKKVDVFVHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRT

SEQ ID NO: 27 - protein, 306 aa, Vibrio parahaemolyticus
VTHYLIGLEENFKRELDGDEWLGYAILPLLATTVSLQITYMACGLDYKDEFGFTDSDVHKLTRNIDKLYDDVSSYITELAAWADNDSYNNANQDNVYDEVMGARSWCTVHGFEHMLIWQKIKELKKVDVFVHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKPSVRLQLEGHFICGMLADQEGSDKVAAFSVAYELFHPDEFGTEK

SEQ ID NO: 28 - protein, 438 aa, Vibrio parahaemolyticus
MTNEYVVTMSSLTEFNPNNARKSYLFDNYEVDPNYAFKAMVSFGLSNIPYAGGFLSTLWNIFWPNTPNEPDIENIWEQLRDRIQDLVDESIIDAINGILDSKIKETRDKIQDINETIENFGYAAAKDDYIGLVTHYLIGLEENFKRELDGDEWLGYAILPLLATTVSLQITYMACGLDYKDEFGFTDSDVHKLTRNIDKLYDDVSSYITELAAWADNDSYNNANQDNVYDEVMGARSWCTVHGFEHMLIWQKIKELKKVDVFVHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKPSVRLQLEGHFICGMLADQEGSDKVAAFSVAYELFHPDEFGTEK

SEQ ID NO: 29 - protein, 111 aa, Vibrio parahaemolyticus
MSNNIKHETDYSHDWTVEPNGGVTEVDSKHTPIIPEVGRSVDIENTGRGELTIQYQWGAPFMAGGWKVAKSHVVQRDETYHLQRPDNAFYHQRIVVINNGASRGFCTIYYH

SEQ ID NO: 30 - DNA, 1124 bp, Artificial
  dsVp19
tgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgtaaaacgacggccagtgccaagcttgcatgcctgcaggtcgacgattctaccgttcgtataatgtatgctatacgaacggtagaatctctagactagtaaaaaaaaaccccgccgaagcggggtttttttttctcggatctcaaaggaggtaaggatcactagaaaattttttaaaaaatctcttgacattggaagggagatatgttattataagaattgctctagattggggtaagacataaaaacggtggcggcggaaacgaggaacagaagagcggacccagccagaagcatcatatccctggtcctgttcttatatttgtcctcatcatcgttatcgttggcagtgtcgtcatcatcatcggtgtccttatcagtgtcagaatcgctgtccttctttggtcccatccatacattcatgacggccaggacgaggataccaattgagatagcaacgatcaaaaagagacccatgcgagccatagacatggagccttcaagatcttccatggtgtaagaagggccagcggcctgggctccggtcctgccgaaaggaagagtgttagtcgtggtggctctagagcaattcttataataacatatctcccttccaatgtcaagagattttttaaaaaattttctagtgatccttacctcctttggaattgatccccgggtaccctagtaaaaaaaaaccccgccgaagcggggtttttttttctccgagctcgaatccgtaatcatggtcatagctgtttcctgatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcc

SEQ ID NO: 31 - RNA, 59 nt, Artificial
  anti-sense RNA sequence targeting expression of dam gene in Vibrio
cuuuuucaucuacugcucuaucuaucgaccaaaaauuaaggcugcggaauguaacauau

(19) United States
(12) Patent Application Publication
(10) Pub. No.: US 2023/0233621 A1
(43) Pub. Date: Jul. 27, 2023

(54) NOVEL PROBIOTIC BACTERIA AND METHODS TO CONTROL PATHOGENS IN AQUATIC ANIMALS

(71) Applicant: Pebble Labs Inc., Los Alamos, NM (US)

(72) Inventors: Elena Sineva, Bernalillo, NM (US); Pedro Costa-Nunes, Albuquerque, NM (US); Tatiana Vinogradova-Shah, Rochester, NY (US)

(21) Appl. No.: 18/001,325

(22) PCT Filed: Jun. 11, 2021
(86) PCT No.: PCT/US2021/037056

§ 371 Date: Dec. 9, 2022

Related U.S. Application Data
(60) Provisional application No. 63/038,304, filed on Jun. 12, 2020.

(51) Int. Cl. A61K 35/742; C12N 1/20; A01N 63/22; A61P 31/20; A23K 10/18; A23K 50/80

(52) U.S. Cl. CPC A61K 35/742; A01N 63/22; A23K 10/18; A23K 50/80; A61P 31/20; C12N 1/205; C12R 2001/125

(57) ABSTRACT

The invention is directed to novel probiotic bacterial strains that colonize animal tissues, and in particular the gastrointestinal (GI) tract of aquatic animals grown in aquaculture environments and may further be engineered to express and deliver interfering RNA molecules configured to downregulate expression of one or more pathogen, or endogenous host genes.

### CROSS-REFERENCE TO RELATED APPLICATIONS

This International PCT Application claims the benefit of and priority to U.S. Provisional Application No. 63/038,304 filed Jun. 12, 2020. The entire specifications and figures of the above-referenced applications are hereby incorporated, in their entirety by reference.

### SEQUENCE LISTING

The instant application contains a Sequence Listing which has been submitted electronically in ASCII format and is hereby incorporated by reference in its entirety. Said ASCII copy, created on Jun. 11, 2021, is named “90115.00560-1-AF.txt” and is 55.4 Kbytes in size.

### DEPOSIT INFORMATION

The bacterial strains MM-W1 (PTA-126789) and MM-W2 (PTA-126790) have been deposited in an international depository under conditions that assure that access to the culture will be available during the pendency of this patent application and any patent(s) issuing therefrom to one determined by the Commissioner of Patents and Trademarks to be entitled thereto under 37 C.F.R. 1.14 and 35 U.S.C. 122. These strains have been deposited in the American Type Culture Collection (ATCC), at 10801 University Boulevard, Manassas, VA., 20110-2209 United States of America.

### TECHNICAL FIELD

The inventive technology generally relates to novel probiotic bacterial strains that may colonize animal tissues, and in particular the gastrointestinal (GI) tract of aquatic animals grown in aquaculture environments and may further be engineered to express and deliver interfering RNA molecules configured to downregulate expression of one or more pathogen, or endogenous host genes.

### BACKGROUND

The development of aquaculture has generated a significant shift in global food production away from traditional catch production methods. Driven primarily by population increases, as well as a lack of growth in traditional capture fishery production, aquaculture has expanded rapidly to become a major component in the world-wide food production eco-system. One major drawback of aquaculture systems is that the aquatic animals are typically placed in high density production systems. This can result in stress from crowding and sub-optimal water quality conditions that provide for easy transmission of disease. In particular, disease outbreaks in aquaculture systems can result in massive losses among aquatic populations, resulting in large economic losses in commercial aquaculture. In the case of shrimp aquaculture, the problem of disease is especially severe. According to the UNFAO, although global aquaculture shrimp production has increased, major producing countries, particularly in Asia, have experienced a significant decline in output as a result of widespread shrimp disease. There are several reasons for this.

First, unlike vertebrates, shrimp lack many of the key components of adaptive and innate immune response mechanisms preventing many traditional methods of inducing or enhancing natural disease resistance. Second, most of the major pathogenic viruses cause very low-level persistent infections that can occur at moderate to very high prevalence in apparently healthy shrimp populations. The majority of shrimp pathogens are transmitted vertically and disease is the result of a massive viral amplification that follows exposure to various forms of environmental or physiological stress. Stressors can include handling, spawning, poor water quality, or abrupt changes in temperature or salinity. Shrimp viruses can also be transmitted horizontally. Once viral loads are high and disease is manifest, horizontal transmission of infection is accompanied by transmission of disease. Third, shrimp commonly are infected simultaneously or sequentially with multiple viruses, or even different strains of the same virus. This fact poses significant challenges for diagnosis, detection, and pathogen exclusion in aquaculture systems.

As one example among many, white spot syndrome (WSS) is a viral disease caused by white spot syndrome virus (WSSV). WSSV is a major pathogen in shrimp that causes high mortality and huge economic losses in shrimp aquaculture. The WSS virion is a nonoccluded ellipsoid- or bacilliform-shaped enveloped particle about 275 nm in length and 120 nm in width. Its circular double-stranded DNA consists of 300 kbp covering approximately 185 open reading frames (ORFs). WSSV is currently one of the most significant impediments to the economical sustainability and growth of the global crustacean aquaculture trade.

Traditional efforts to prevent and treat shrimp pathogens, such as WSSV, have been met with limited success. For example, attempts to reduce environmental and physiological stressors has been limited due to the economic production needs of aquaculture systems as well as a lack of technical expertise and appropriate aquaculture facilities in many developing countries. Other attempts have been made to create and isolate pathogen-free populations for aquaculture. However, such efforts are slow and require significant expertise and diagnostic capabilities that are prohibitively expensive. Large-scale applications of antibiotics have been applied to shrimp aquaculture, in particular during the production cycle, both in the larval and growth phases. However, the use of antibiotics in aquaculture has been associated with environmental and human health problems, including bacterial resistance, and persistence of the disease in the aquatic environment. The accumulation of antibiotic residues in the edible tissues of shrimp may also alter human intestinal flora and cause food poisoning or allergy problems. Most importantly, antibiotics are ineffective against viruses. Other methods such as the application of immunostimulants or bacteriophage treatments to target specific pathogens have been tried with limited commercial and practical success.

As such, there exists a need for improved strategies for controlling pathogens in aquatic animals raised in aquaculture and other animal systems. As will be discussed in more detail below, the current inventive technology includes the novel use enteric bacteria to provide a vehicle for stable and continuous non-integrative transformation of a target host cell through the continual delivery of select molecules, such as RNA molecules configured to induce an endogenous RNA interference response, as well as mRNAs produced in enteric bacteria that are configured to be translated in the eukaryotic target host, and preferably an aquatic animal target host such as shrimp grown in aquaculture.

### SUMMARY OF THE INVENTION

The current invention includes isolation, identification, and modification of novel enteric bacteria, and preferably novel probiotic enteric bacteria that may be a probiotic for aquatic animals such as shrimp. In one preferred embodiment, the current invention includes isolation, identification, and modification of novel enteric probiotic bacteria, and specifically Bacillus Subtilis sp. MM-W1 and MM-W2 as generally described herein.

Additional embodiments of the invention may include one or more of the following preferred embodiments:

1. A bacterial strain according to MM-W1.

2. A bacterial strain according to MM-W2.

3. The bacterial strain of any of embodiments 1 and 2, wherein the bacterial strains are genetically engineered to express a heterologous nucleotide sequence, operably linked to a promoter, encoding a eukaryotic-like mRNA that may be introduced to and translated in a target host.

4. The bacterial strain of any of embodiments 1 and 2, wherein the bacterial strains are genetically engineered to express a heterologous nucleotide sequence, operably linked to a promoter, encoding an inhibitory RNA molecule configured to inhibit expression of at least one of the following:
at least one endogenous gene in a target host;
at least one gene of a target host pathogen; and
at least one gene involved in quorum sensing and/or biofilm formation; and

5. The bacterial strain of embodiment 1, wherein the bacterial strains are genetically engineered to express a heterologous nucleotide sequence, operably linked to a promoter, encoding one or more of the following:
a heterologous double stranded RNA (dsRNA) molecule configured to inhibit expression of at least one target gene; and
a heterologous engineered RNaseIII configured for enhanced generation of small interfering RNAs (siRNAs) from catalytic cutting of said heterologous dsRNA molecule.

6. The bacterial strain of embodiment 5, wherein said target gene comprises at least one of the following:
at least one endogenous gene in a target host;
at least one gene of a target host pathogen; and
at least one gene involved in quorum sensing and/or biofilm formation.

7. The bacterial strain of any of embodiments 1 and 2, wherein the bacterial strains are genetically engineered to express a heterologous truncated toxin peptide configured to competitively inhibit the activity of a target toxin produced by an EMS-causing bacterial pathogen.

8. The bacterial strain of any of embodiments 1-7, wherein said target host comprises an aquatic animal.

9. The bacterial strain of embodiments 8, wherein said aquatic animal comprises a shrimp.

10. The bacterial strain of any of embodiment 4, and 6, wherein said target host pathogen comprises a pathogen selected from the group comprising: white spot syndrome virus (WSSV), Early Mortality Syndrome (EMS) causing bacteria, a Vibrio bacterial pathogen, and quorum sensing (QS) in pathogenic bacteria.

11. The bacterial strain of any of embodiment 4, 6, and 10, wherein said gene of a target host pathogen is selected from the group of WSSV genes consisting of: the vp28 gene, the vp19 gene, or the wsv477 gene.

12. The bacterial strain of any of embodiment 4, 6, and 11, wherein said inhibitory RNA molecule is selected from the group consisting of: SEQ ID NO.’s 1, 2, 3 and 30.

13. The bacterial strain of embodiment 10, wherein said gene of a target host pathogen is selected from the group of Vibrio genes consisting of: DNA adenine methylase (dam) gene

14. The bacterial strain of any of embodiment 4, 6, and 13, wherein said inhibitory RNA molecule is selected from the group consisting of: SEQ ID NO. 22, and/or SEQ ID NO. 31.

15. The bacterial strain of any of embodiments 1 and 2, wherein the bacterial strains are genetically engineered to express a heterologous nucleotide sequence, operably linked to a promoter, encoding a heterologous quorum quenching molecule configured to remove exogenous AI-2 molecules from the environment and transport them into said bacterial strain

16. The bacterial strain embodiment 15, wherein said heterologous quorum quenching molecule comprises a heterologous lsr operon operably linked to a promotor configured to express at least one ATP -binding cassette transporter (ABC transporter) and at least one chaperone protein that actively pump exogenous autoinducer molecule AI-2 from the environment into said transformed bacterial cell.

17. The bacterial strain of embodiment 16, wherein said heterologous lsr operon is identified as SEQ ID NO. 20.

18. The bacterial strain embodiment 15, wherein said heterologous quorum quenching molecule comprises a homoserine lactonases (AHL lactonase) configured to inactivate exogenous AI-1 molecules.

19. The bacterial strain embodiment 18, wherein said AHL lactonase comprises a heterologous aidH gene is identified as SEQ ID NO. 21.

20. The bacterial strain of embodiment 7, wherein said EMS-causing bacterial pathogen comprises a pathogenic species of Vibrio.

21. The bacterial strain of embodiment 20, wherein said target toxin produced by an EMS-causing bacterial pathogen comprises a Pir toxin.

22. The bacterial strain of embodiment 20, wherein said Pir toxin comprises a PirA peptide according to amino acid sequence identified as SEQ ID NO. 29, and a PirB peptide according to amino acid sequence identified as SEQ ID NO. 28.

23. The bacterial strain of embodiment 22, wherein said truncated toxin peptide configured to competitively inhibit the activity of a target toxin produced by an EMS-causing bacterial pathogen comprises a truncated PirB peptide, and a truncated PirA peptide.

24. The bacterial strain of embodiment 23, wherein said truncated PirB peptide comprises a truncated PirB peptide having a portion of its C-terminal domain removed.

25. The bacterial strain of embodiment 24, wherein said truncated PirB peptide having a portion of its C-terminal domain removed comprises a truncated according to the according to amino acid sequence identified as SEQ ID NOs. 24-26.

26. The bacterial strain of embodiment 25, wherein said truncated PirB peptide comprises a truncated PirB peptide having a portion of its N-terminal domain removed.

27. The bacterial strain of embodiment 26, wherein said truncated PirB peptide having a portion of its N-terminal domain removed comprises a truncated according to the according to amino acid sequence identified as SEQ ID NO. 27.

28. The bacterial strain of embodiment 26, wherein said truncated PirB peptide comprises a truncated PirB peptide having one or more domain-disrupting modifications.

29. The bacterial strain of embodiment 3, wherein said eukaryotic-like mRNA comprises a eukaryotic-like mRNA configured to generate a phenotypic change in said target host.

30. The bacterial strain of embodiment 29, wherein said phenotypic change comprises a phenotypic change selected from the group consisting of: increased growth; enhanced stress resistance; enhanced disease resistance; production of a naturally occurring compounds; production of a non-naturally occurring compounds; therapeutic pathogen bio-control; reduction in disease condition; a gene editing function.

31. The method of embodiment 30, wherein the gene editing function comprises a gene-editing endonuclease selected from the group consisting of: CRISPR-associated endonuclease, Cas-9, Cas-3, a TALAN-associated endonuclease; a meganuclease, and a zinc-finger associated endonuclease.

32. The bacterial strain of embodiment 5, wherein the siRNAs generated by the engineered RNaseIII are between 22-23 nucleotides in length.

33. The bacterial strain of embodiment 32, wherein said engineered RNaseIII comprises an engineered RNaseIII having the following mutations: E38A/R107A/R108A.

34. The method of any of embodiments 5, 32 and 33, wherein said engineered RNaseIII comprises an engineered RNaseIII selected from the group consisting of: the nucleotide sequence according to SEQ ID NOs. 4, 8, and 12, and amino acid sequences SEQ ID NOs. 5, 9, and 13.

35. Administering a therapeutically effective amount of the bacterial strain of any of the embodiments above to an aquatic animal in need thereof.

36. A method of treating WSSV, comprising administering a therapeutically effective amount of the bacterial strain of any of the embodiments above to a shrimp in need thereof.

37. A method of treating EMS, comprising administering a therapeutically effective amount of the bacterial strain of any of the embodiments above to a shrimp in need thereof.

38. A method of preventing biofilm formation, comprising administering a therapeutically effective amount of the bacterial strain of any of the embodiments above to a shrimp in need thereof.

39. A method of preventing biofilm formation, comprising administering a therapeutically effective amount of the bacterial strain of any of the embodiments above to an aquaculture environment.

40. The bacterial strain of any of the embodiments above wherein said nucleotide sequence is codon optimized for expression in Bacillus Subtilisname>.

41. The bacterial strain of any of the embodiments above wherein said bacterial strain is incorporated into a feed for aquatic animals.

Additional embodiments of the invention may become evident in light of the figures and disclosure provided below.

### BRIEF DESCRIPTION OF THE FIGURES

FIGS. 1A-B. Integration constructs. (A) An integration construct are long PCR fragments consistent of several parts. (B) Example of integration construct assembly and verification of the results by colony PCR.

FIGS. 2A-B. (A) Plasmid map of pSC-Pgrac-cre. (B) Scheme of antibiotic marker removal using pSC-Pgrac-cre.

FIGS. 3A-B. (A) Plasmid map of pAD-2grac100-vp19. (B) Comparison of dsRNA-vp19 expression directed by Pupp and Pgrac promoters. Expression of dsRNA-vp19 was determined by RT-qPCR using Luna® Universal One-Step RT-qPCR Kit (NEB).

FIGS. 4A-C. Integration of dsRNA-Vp19 expression cassette into Bacillus genome leads to the better protection of shrimp against WSSV infection. (A) Overall design of Vp19 expression cassettes (B) Relative expression of vp19 RNA in plasmid based and integration constructs. (C) Effect of shrimp feed supplement with antiviral constructs on shrimp survival during WSSV infection trials.

FIG. 5. High molecular and low molecular RNA accumulation in MM-W1 and MM-W2. RNaseIII N3XT expression leads for vp19 smRNA formation. Knock-out of Bacillus rnc is important to both dsRNA accumulation and smRNA formation.

### DETAILED DESCRIPTION OF THE INVENTION

One embodiment of the current invention includes isolation, identification, and modification of novel enteric bacteria, and preferably novel probiotic enteric bacteria that may be a probiotic for aquatic animals such as shrimp. In one preferred embodiment, the current invention includes isolation, identification, and modification of novel enteric probiotic bacteria, and specifically Bacillus Subtilis sp. MM-W1 and MM-W2 as generally described herein.

The inventive technology may include compositions and methods of use for the probiotic enteric bacterial strains of Bacillus Subtilis, and in particular B. Subtilis sp. MM-W1 and/or MM-W2. Notably, B. Subtilis is a probiotic bacterium that is generally regarded as safe (GRAS) by the FDA. To create therapeutic constructs expressing an exemplary inhibitory RNA molecule, in this case a dsRNA, the present inventors initially identified B. Subtilis Δ6 strain that is available out of Bacillus Genetic Stock Center. Notably, B. Subtilis Δ6 strain has had all prophages deleted from its genome. Because of these deletions, the genome size of strain B. Subtilis Δ6 has been reduced 8.1% relative to the 168 parent. As discussed below, deletion of prophages are prerequisite to the knockout of RNaseIII that degrades dsRNA in all bacterial cells. This deletion allows for the efficient production and delivery of dsRNA by B. Subtilis sp. MM-W1 and/or MM-W2 as shown below.

In one embodiment, B. Subtilis we used before to create plasmid-based constructs expressing dsRNA identified as SEQ ID NO. 1-3, or 30. expression of these dsRNA is directed by a strong Pgrac promoter that increases RNA production by several order of magnitude over traditional promoters, which allows the dsRNA expression cassette to be stably integrated into bacterial genome without losing dsRNA producing potency. In absence of RNaseIII bacterial cells produce long (~300 bp) dsRNA. These dsRNA may be processed by animal host cells to fragments of 22-23 bp siRNA that may be bound up by the host’s RISC system to silence target gene expression, and in this case target WSSV genes: viral capsid protein 19 (vp19); viral capsid protein 19 gene (vp28); and early non-structural gene 477 (Wsv477).

To create a DICER-independent RNAi enabled bacteria that produces pre-processed RNA, the present inventors introduced into the Bacillus genome an E38A-R107A-R108A RNaseIII (generally referred to as RNaseIIIN3XT) mutant expression cassette by integration into the neutral ywdD locus. The E38A-R107A-R108A RNaseIII mutant (nucleotide SEQ ID NOs. 4, 8, and 12, and amino acid sequences SEQ ID NOs. 5, 9, and 13) has improved catalytic efficiency and enhanced dsRNA cutting specificity for 22 and 23 nt sRNAs. In this manner, the RNaseIII N3XT may generate 22 and 23 nt sRNAs from the dsRNA expressed in B. Subtilis sp. MM-W1 and/or MM-W2 that may be delivered to a host and bound by the RISC complex, independent of DICER, to downregulate expression of a target pathogen gene, such as the vp19 gene of WSSV. The present inventor further removed any antibiotic resistance marks or toxin genes and further deleted the wt rnc (RNaseIII.)

As a result, the present inventors have constructed two Bacillus strains MM-W1 and MM-W2, that have no detectable antibiotic resistance markers or toxin genes, have wt rnc (RNaseIII) deleted, and have stably integrated cassettes expressing dsRNA complementary to WSSV viral capsid 19 gene. MM-W1 further expresses an RNaseIIIN3XT construct encoding a E38A-R107A-R108A RNaseIII. Strain genotypes are shown in Table 1 below.

Another embodiment of the current invention related to novel enteric bacteria that may be used in methods to control pathogens, and preferably pathogens in aquatic animals, such as shrimp. In one preferred embodiment, the present invention may include novel genetically modified enteric bacteria, or novel bacterial combinations, which can be engineered to express one or more heterologous inhibitory RNA molecules that may be delivered to the target host.

In another embodiment, the inventive technology may include compositions and methods of use for the bacterial strains B. Subtilis sp. MM-W1 and B. Subtilis sp. MM-W2. In one embodiment, probiotic bacterial strains MM-W1 and MM-W2 may be genetically engineered to express a heterologous nucleotide sequence, operably linked to a promoter sequence, encoding an inhibitory RNA molecule. In this preferred embodiment, enteric probiotic bacterial strains MM-W1 and MM-W2 8 may be genetically engineered to express a heterologous nucleotide sequence, operably linked to a promoter sequence, encoding one or more double stranded RNA (dsRNA) molecules directed to initiate a DICER-mediated RNA interference (RNAi) response causing the destruction of specifically targeted pathogen mRNA molecules that may be present within a target host. In further embodiments, the enteric probiotic bacteria such as B. Subtilis sp. MM-W1 and MM-W2 may colonize in the gut of a target host such as a shrimp, such that the dsRNAs are continually delivered to the target host.

In another embodiment, the present invention may include novel genetically modified enteric probiotic bacteria, or novel bacterial combinations, which can be used to deliver one or more heterologous dsRNA molecules thereby initiating an RNAi response in a target host. In one preferred embodiment, the enteric probiotic B. Subtilis sp. MM-W1 and MM-W2, may be configured to express a heterologous dsRNA configured to inhibit expression of one or more essential genes of a host pathogen. The enteric probiotic B. Subtilis sp. MM-W1 and MM-W2 may be further a DICER-independent RNAi systems, whereby the bacteria are configured to express a heterologous engineered RNase III enzyme configured to processes double-strand RNA (dsRNA) almost exclusively into small RNAs (sRNAs) comprising of 22 and 23 nt. These sRNAs may be transported from the bacterial strains and initiate a DICER-independent RNAi response in a target host, such as a shrimp. In one preferred embodiment, the engineered RNase III may include an engineered RNase III having the following mutations E38A/R107A/R108A (RNase III N3XT™) which was demonstrated to processes double-strand RNA (dsRNA) almost exclusively into sRNAs (sRNAs) comprising of 22 and 23 nt (such systems, methods and compositions, being included in PCT/US2019/025261, the specification, sequences 1-121, examples, and figures related to the generation of siRNAs in a DICER independent manner are herein incorporated in their entirety by reference). In this embodiment, enteric probiotic B. Subtilis sp. MM-W1 and MM-W2 may be further be genetically modified to include a knockout of the wild type RNase III (Δrnc).

In another embodiment, B. Subtilis sp. MM-W1 and MM-W2 may be engineered to express one or more dsRNA molecules targeting one or more essential genes in a host viral pathogen, such as WSSV. Examples of such essential genes and dsRNA directed to said essential genes for the treatment and prevention of WSS being generally described in PCT/US2018/025766, the specification, figures, and sequences SEQ ID NO. 1-10 being specifically incorporated herein by reference). In this embodiment, the probiotic enteric B. Subtilis sp. MM-W1 and MM-W2 can be useful for the treatment of target hosts, such as shrimp and other aquatic animals, which are susceptible to such viral pathogens. In another embodiment, B. Subtilis sp. MM-W1 and MM-W2 expressing an dsRNA or anti-sense RNA targeting one or more essential genes in a host pathogen, such as WSSV, and more specifically, viral capsid protein 19 (vp19), and/or viral capsid protein 19 gene (vp28) and/or early non-structural gene 477 (Wsv477), and may further express an engineered RNaseIII mutant as noted above having the following mutations E38A/R107A/R108A can be useful for the treatment of target hosts, such as shrimp and other aquatic animals, which are susceptible to such viral pathogens in a DICER-independent manner.

In another embodiment, B. Subtilis sp. MM-W1 and MM-W2 may be engineered to express one or more anti-sense RNA molecules (asRNA) targeting one or more essential genes in a host bacterial pathogen, such as pathogenic species of Vibrio that may cause, for example, Acute Hepatopancreatic Necrosis Disease A/K/A Early Mortality Syndrome (EMS). Examples of such essential genes and asRNA directed to said essential genes for the treatment and prevention of EMS being generally described in PCT/US2018/033976, the specification, figures, and sequences SEQ ID NOs. 1-4 being specifically incorporated herein by reference). In this embodiment, the probiotic enteric B. Subtilis sp. MM-W1 and MM-W2 can be useful for the treatment of target hosts, such as shrimp and other aquatic animals, which are susceptible to such bacterial pathogens.

In another embodiment, B. Subtilis sp. MM-W1 and MM-W2 may be engineered to express one or more anti-sense RNA molecules (asRNA) targeting one or more essential genes in a host bacterial pathogens, such as pathogenic species of Vibrio that may be involved in bacterial quorum sensing, the formation of biofilms, as well as conditions such as EMS. Examples of such essential genes and asRNA directed to said essential genes being generally described in PCT/US2018/045687, the specification, figures, and sequences SEQ ID NOs. 1-4 being specifically incorporated herein by reference). In this embodiment, the probiotic enteric B. Subtilis sp. MM-W1 and MM-W2 can be useful for the treatment of target hosts, such as shrimp and other aquatic animals, which are susceptible to bacterial biofilm formation.

In another embodiment, B. Subtilis sp. MM-W1 and MM-W2 may be engineered to express one or more heterologous dsRNA molecules thereby initiating an RNAi response directed to an endogenous gene in a target host. In one preferred embodiment, B. Subtilis sp. MM-W1 and MM-W2 may, may be further configured to express a heterologous engineered RNaseIII enzyme configured to processes double-strand RNA (dsRNA) almost exclusively into small RNAs (sRNAs) comprising of 22 and 23 nt. These sRNAs may be transported from the bacterial strains and initiate a DICER-independent RNAi response directed to an endogenous gene in a target host.

In another embodiment, the present invention may include probiotic enteric bacteria, or bacterial combinations, that may be engineered to express one or more RNA transcripts that may be delivered and translated in a target host. In one preferred embodiment, probiotic enteric bacteria, and preferably B. Subtilis sp. MM-W1 and MM-W2 may be configured to produce eukaryotic-like mRNA that may be introduced to, and translated in a target host such as a shrimp, for example as generally described by Sayre et al in PCT/US2019/040747 (the specification, figures, and sequences SEQ ID NOs. 1-37 being specifically incorporated herein by reference). In one preferred embodiment, such mRNAs expressed in B. Subtilis sp. MM-W1 and MM-W2 and translated it the host may include one or more endonucleases, such as cas-9 or -3, as well as additional components such as a guide RNA (gRNA) directed to a target sequence in the host.

Another embodiment of the invention may include methods or treating or preventing infection in a target host by administering a therapeutically effective amount of B. Subtilis sp. MM-W1 and MM-W2 to the target host, wherein B. Subtilis sp. MM-W1 and MM-W2 expresses at least one inhibitory RNA molecules, such as a dsRNA or asRNA or both directed to an essential pathogen gene or host endogenous gene.

Another embodiment of the invention may include methods or treating or preventing infection by WSSV or a pathogenic species of Vibrio in a target host by administering a therapeutically effective amount of B. Subtilis sp. MM-W1 and MM-W2 to the target host, wherein B. Subtilis sp. MM-W1 and MM-W2 expresses at least one inhibitory RNA molecules, such as a dsRNA or asRNA or both directed to an essential gene in WSSV or a pathogenic species of Vibrio or host endogenous gene.

Another embodiment of the invention may include methods or treating or preventing bacterial biofilms in a target host by administering a therapeutically effective amount of B. Subtilis sp. MM-W1 and MM-W2 to the target host, wherein B. Subtilis sp. MM-W1 and MM-W2 expresses at least one inhibitory RNA molecules, such as a dsRNA or asRNA or both directed to an essential pathogen gene or host endogenous gene involved in biofilm formation and/or quorum sensing.

Another embodiment of the invention may include the isolated bacteria B. Subtilis sp. MM-W1 and MM-W2. Another embodiment of the invention may include the incorporation of isolated bacteria B. Subtilis sp. MM-W1 and/or MM-W2 in an animal feed, and preferably a feed for aquatic animals. In one preferred embodiment, B. Subtilis sp. MM-W1 and/or MM-W2 be delivered as a top coating on feed of aquaculture animals. In yet another embodiment, B. Subtilis sp. MM-W1 and/or MM-W2 may also be self-eliminating from the aquatic animal, such as a shrimp, before harvest due to a natural or engineered auxotrophy, in this case MM-W1 and MM-W2 are tryptophan auxotrophs (trpC2). In this embodiment, due to a natural or engineered auxotrophy, B. Subtilis sp. MM-W1 and/or MM-W2 may not persist in the environment due to inability to grow without specific compounds present in the feed.

As noted above, in a preferred embodiment, one or more inhibitory RNA molecules, in this instance dsRNA, may be delivered to a target host/population of shrimp through MM-W1 and/or MM-W2 that may colonize in the gut of the shrimp. In this embodiment, once colonized in the host, vertical transmission of MM-W1 and/or MM-W2 may be passed to the host’s progeny, thus naturally replicating the pathogenic resistance to subsequent generations. In certain embodiments, MM-W1 and/or MM-W2 expressing one or more inhibitory RNA molecules may colonize a shrimp throughout its lifecycle. For example, MM-W1 and/or MM-W2 expressing one or more inhibitory RNA molecules may colonize a shrimp while it is: an egg, a nauplius, a protozoea, a mysis, post-larval stage or an adult. In this embodiment, the colonized bacteria may express inhibitory RNA molecules, such as dsRNA as generally described or incorporated herein, that may further be processed by the host’s DICER/RISC complex allowing pathogen-specific mRNA silencing/inactivation of essential pathogen genes, or through a co-expresses heterologous RNAseIII-N3XT genetic construct that may allow a DICER independent generation of sRNAs.

Moreover, MM-W1 and/or MM-W2 may be auxotrophic such that their colonization of the gut microbiome may be cleared in the absence of the one or more nutrients, such as tryptophan autotrophy that may be supplemented directly to the shrimp population, for example through a feed. In this embodiment, during colonization, MM-W1 and/or MM-W2 may continuously deliver the dsRNA molecules via the intestine from the earliest larval stages to the adult stage, providing pathogen-specific mRNA silencing/inactivation of essential pathogen genes throughout the host’s lifecycle. In addition, having been cleared prior to harvest, MM-W1 and/or MM-W2 may not pose any risk to the organism, environment, or end-consumers.

Additionally, in certain embodiments MM-W1 and/or MM-W2 may also be horizontally transmitted to a host population through the distribution of MM-W1 and/or MM-W2 in treated feed, or feed containing the MM-W1 and/or MM-W2, or propagation of the MM-W1 and/or MM-W2 bacteria into the environment as excreted animal waste. Such a feature may allow for the one-time or at least only periodic administration of the modified bacteria to the host and/or host’s environment generating a significant commercial advantage. The inventive technology may further comprise methods and techniques to control the levels and timing of the expression of inhibitory RNA molecules in the target organism.

Thus, according to one embodiment of the present invention, there is provided a method of controlling a pathogenically infected shrimp, the method comprising administering to a shrimp population MM-W1 and/or MM-W2 expressing a heterologous nucleic acid sequence which specifically downregulates an expression of at least one essential pathogen gene product of the shrimp, wherein downregulation of the expression of the at least one essential pathogen resistance gene may prevent replication and/or pathogenicity of the shrimp pathogen. In one preferred embodiment, the pathogen may include WSSV or a pathogenic species of Vibrio.

In one preferred embodiment, the invention may include methods and compositions for the biocontrol of WSSV infection in shrimp. In this preferred embodiment, a shrimp population may be administered a genetically modified bacteria expressing a heterologous nucleic acid sequence which specifically downregulates an expression of at least one essential WSSV gene product, wherein downregulation of the expression of the essential WSSV gene may prevent replication and/or pathogenicity of WSSV in shrimp.

In this preferred embodiment, the heterologous nucleic acid sequence expresses an RNA duplex, comprising a sense region and an antisense region, wherein the antisense region includes a plurality of contiguous nucleotides that are complementary to a messenger RNA sequence encoded by the target gene. In one embodiment, the polynucleotide encoding the siRNA comprises at least one nucleotide sequence configured to generate a hpRNA that targets one or more essential WSSV genes. In this preferred embodiment, such hpRNAs may inhibit expression of target genes in WSSV including, but not limited to: viral capsid protein 19 (vp19), viral capsid protein 19 (vp28), and early non-structural gene (Wsv477) among others.

In this embodiment, a heterologous nucleic acid sequence expresses an RNA duplex, or hpRNA, may be selected from the group consisting of: SEQ ID NO. 1, SEQ ID NO. 2, SEQ ID NO. 3, and/or SEQ ID NO. 30. In this embodiment, SEQ ID NO. 1 is configured to target and inhibit expression of an early non-structural gene (Wsv477)(SEQ ID NO. 18 and 18). SEQ ID NO. 2 is configured to target and inhibit expression of viral capsid protein 28 (vp28) (SEQ ID. NO 16 and 17), and SEQ ID NO. 3, and/or SEQ ID NO. 30 are configured to target and inhibit expression of viral capsid protein 19 (vp19) (SEQ ID. NO. 14 and 15). It should be noted that the identification of a DNA sequence also includes the corresponding RNA sequence it encodes. As such, a reference to a SEQ ID NO. that includes DNA also specifically include the sequence of the RNA that it expresses as would be understood by one of ordinary skill in the art. For example, where it is claims that a heterologous inhibitory polynucleotide may be selected from the group consisting of: SEQ ID NO. 1, SEQ ID NO. 2, SEQ ID NO. 3, and/or SEQ ID NO. 30, such a claim may include the sequence of the inhibitory RNA molecule as one of ordinary skill could easily determine without undue experimentation.

In a preferred embodiment, a messenger RNA sequence encoded by the target pathogen gene may include a gene located in a region of higher than average homology, or in other words, a gene fully or partially located in the most conserved region of a pathogens genome, when compared to the sequences of other strains of the pathogens of genes. In one specific embodiment, SEQ ID NO. 1, SEQ ID NO. 2, SEQ ID NO. 3, and/or SEQ ID NO. 30 correspond to highly conserved structural and/or non-structural proteins coding regions in SEQ ID NO. 14-19, generally. Naturally, such sequences are exemplary, as they may be alternatively, redundant or overlapped across one or more distinct gene coding segments.

In another embodiment, MM-W1 and/or MM-W2 may express at least one heterologous quorum quenching molecule configured to remove exogenous AI-2 molecules from the environment and transport. In a preferred embodiment of the invention, MM-W1 and/or MM-W2 may express a heterologous lsr operon operably linked to a promotor that is configured to express at least one ATP -binding cassette transporter (ABC transporter) and at least one chaperone protein that actively pump exogenous autoinducer molecule AI-2 from the environment into said MM-W1 and/or MM-W2 cell. In this embodiment, the lsr operon is identified as SEQ ID NO. 20.

In another preferred embodiment, MM-W1 and/or MM-W2 may express a heterologous acylated homoserine lactonases (AHL lactonase), which may aidH gene from Ochrobactrum identified as SEQ ID NO. 21. In one preferred embodiment, such a nucleic acid agent may include an asRNA polynucleotide identified as SEQ ID NO. 22, and/or SEQ ID NO. 31. Additional embodiments may include any nucleic acid that spans a region of greater than average homology between an essential target gene of various strains of an EMS-causing pathogen. One preferred embodiment may include any nucleic acid that spans a region of greater than average homology between the essential target genes of various strains of a Vibrio. In the example of an EMS-causing Vibrio disease causing agent, this may include, as shown generally below in the region encoding the dam gene identified as SEQ ID NO. 23, among others. As noted elsewhere, dam is an essential gene in Vibrio sp. and is involved in regulation of gene expression. Dam is also involved in regulation of virulence pathway in many EMS-causing bacteria.

In another preferred embodiment, the invention may include the delivery of one or more asRNA molecules pathogenic bacteria in a host organism, and preferably an aquatic organism such as shrimp, and preferably shrimp raised in aquaculture. In a preferred embodiment, MM-W1 and/or MM-W2 may be configured to deliver one or more asRNA molecules identified as SEQ ID NO. 22, and/or SEQ ID NO. 31, or a sequence having 70% to 99% homology thereof, directed to inhibit dam gene expression, identified as SEQ ID NO. 3, SEQ ID NO. 30 or a sequence having 70% to 99% homology thereof, in EMS-causing pathogenic bacteria in an aquatic host organism, such as shrimp or other organisms commonly raised through aquaculture.

In one embodiment, the present invention may include methods of administering a therapeutically effective amount of MM-W1 and/or MM-W2, configured to express heterologous dsRNA or asRNA polynucleotide that may target an essential target gene in a pathogen, such as WSSV or Vibrio, or genes involved in the formation of biofilms as generally described herein.

In one embodiment, inhibitory RNA molecules directed toward a plurality of pathogen genes and/or endogenous genes may be included in a single construct and may further be expressed in MM-W1 and MM-W2.

In another embodiment, the inventive technology may include novel systems, methods, and compositions for treating and/or preventing Early Mortality Syndrome (EMS) associate mortality susceptible organisms, through the use of MM-W1 and/or MM-W2 to express and deliver to a target host a truncated PirB toxin protein that competitively inhibits and/or deactivates toxins produced of pathogenic Vibrio sp. that cause EMS. MM-W1 and/or MM-W2 may include a heterologous nucleotide sequence, operably linked to a promoter, configured to express a modified or mutated PirB or PirA peptide. In one preferred embodiment, MM-W1 and/or MM-W2 expressing a truncated PirB toxin, wherein the N- or C-terminal domain of the PirB has been removed or disrupted. In this embodiment, the removal or disruption of the C-terminal domain of the truncated PirB toxin may inactivate PirA/PirB toxin activation by both competitive inhibitions of toxin-specific receptors on hepatopancreas cells; and by preventing the formation of pirA/pirB complexes by binding pirA molecules with truncated PirB C-domain. In addition, the removal or disruption of the N-terminal domain of the truncated PirB toxin may prevent the pirA/pirB complexes pore-forming activity.

In one embodiment, MM-W1 and/or MM-W2 may contain one or more genetic constructs that may result in the overexpression of a heterologous truncated PirB or A peptide in the target host. In this embodiment, MM-W1 and/or MM-W2 may be introduced to a target shrimp or aquaculture shrimp population and become a part of the host’s natural microbiome, for example through a feed as generally described herein. The overexpression of the heterologous truncated PirB or A peptide in the target host may cause inactivation of Pir toxin generated by EMS pathogens, such as the various species of Vibrio identified herein.

Additional embodiments of the invention may include MM-W1 and/or MM-W2 encoding one or more truncated PirB peptides identified having a deletion at the C-terminal regions identified as SEQ ID NOs. 24-26. Additional embodiments of the invention may include isolated amino acid sequences encoding a truncated PirB peptide identified having a deletion at the N-terminal regions identified as SEQ ID NO. 27. Additional embodiments of the invention may include isolated amino acid sequences encoding a PirB peptide identified as SEQ ID NO. 28, having one or more domain-disrupting modifications at one or more of the following domains: aa116-aa132, aa214-aa226, aa290-aa300, aa322-aa330, aa386-aa401, aa409-aa421, or aa426-aa434.

Additional embodiments of the invention may include an expression cassette encoding one or more sequences for a truncated PirA peptide identified having a deletion at the C-terminal region operably linked to a promoter. Additional embodiments of the invention may include an expression cassette for a truncated PirA peptide identified having a deletion at the N-terminal regions operably linked to a promoter in MW-W1 and/or MW-W2 In another preferred embodiment, a nucleotide sequence encoding a truncated PirA according to SEQ ID NO. 29, which may be operably linked to a promoter and introduced to a target host, such as a shrimp, through an expression vector which may be MW-W1 and/or MW-W2 having one or more mutations in one or more of the following domains: aa15-aa40, or aa52-aa79.

The term “aquaculture” as used herein includes the cultivation of aquatic organisms under controlled conditions.

The term “aquatic organism” and/or “aquatic animal” as used herein include organisms grown in water, either fresh or saltwater. Aquatic organisms/animals includes vertebrates, invertebrates, arthropods, fish, mollusks, including, shrimp (e.g., penaeid shrimp, Penaeus esculentu, Penaeus setiferus, Penaeus stylirostris, Penaeus occidentalis, Penaeus japonicus, Penaeus vannamei, Penaeus monodon, Penaeus chinensis, Penaeus aztecus, Penaeus duorarum, Penaeus indicus, and Penaeus merguiensis, Penaeus californiensis, Penaeus semisulcatus, Penaeus monodon, brine shrimp, freshwater shrimp, etc), crabs, oysters, scallop, prawn clams, cartilaginous fish (e.g., sea bream, trout, bass, striped bass, tilapia, catfish,, salmonids, carp, catfish, yellowtail, carp zebrafish, red drum, etc), crustaceans, among others. Shrimp include, shrimp raised in aquaculture as well.

The term “probiotic” refers to a microorganism, such as bacteria, that may colonize a host for a sufficient length of time to deliver a therapeutic or effective amount of an interfering RNA molecule. A probiotic may include endosymbiotic bacteria, or naturally occurring flora that may permanently to temporarily colonize an animal, such as an aquatic organism. Probiotic organisms may include MM-W1 and MM-W2 as described in Table 1.

As used herein, the phrase “host” or “target host” refers to an animal carrying a disease-causing pathogen, an animal susceptible to a disease-causing pathogen, an animal population where members are carrying a disease-causing pathogen, or an animal population where members are susceptible to a disease-causing pathogen. In one preferred embodiment, a target host is an aquatic organism, and more preferably a shrimp.

As used herein, the phrase “feed” or “feed” refers to animal consumable material introduced as part of the feeding regimen or applied directly to the water in the case of aquatic animals. A “treated feed” refers to a feed treated with a MM-W1 and/or MM-W2.

As used herein, the term “controlling” and/or “bio-control” refers to reducing and/or regulating pathogen/disease progression and/or transmission.

As used herein and in the appended claims, the singular forms “a”, “an”, and “the” include plural referents unless the context clearly dictates otherwise. Thus, for example, reference to “a plant” includes a plurality of such plants; reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ± a range of up to 20%, up to 15%, up to 10%, up to 5%, or up to 4%, 3%, 2%, or 1% compared to the specifically recited value.

The term “comprising” as used in a claim herein is open-ended and means that the claim must have all the features specifically recited therein, but that there is no bar on additional features that are not recited being present as well. The term “comprising” leaves the claim open for the inclusion of unspecified ingredients even in major amounts. The term “consisting essentially of in a claim means that the invention necessarily includes the listed ingredients and is open to unlisted ingredients that do not materially affect the basic and novel properties of the invention.

“RNase III” refers to a naturally occurring enzyme or its recombinant form. The RNase III family of dsRNA-specific endonucleases is characterized by the presence of a highly conserved 9 amino acid stretch in their catalytic center known as the RNaseIII signature motif. Mutants and derivatives are included in the definition. The utility of bacterial RNase III described herein to achieve silencing in mammalian cells further supports the use of RNases from eukaryotes, prokaryotes viruses or archaea in the present embodiments based on the presence of common characteristic consensus sequences. The designations for the mutants are assigned by an amino acid position in a particular RNaseIII isolate. These amino acid positions may vary between RNase III enzymes from different sources. For example, E38 in E. coli corresponds to E37 in Aquifex aeolicus. The positions E38 in E. coli and E37 in A. aeolicus correspond to the first amino acid position of the consensus sequence described above and determined by aligning RNaseIII amino acid sequences from the public databases by their consensus sequences. Embodiments of the invention are not intended to be limited to the actual number designation. Preferred embodiments refer to relative position of the amino acid in the RNaseIII consensus sequence(s). In particular, the invention includes residues 38, 65, 107 and 108 and their corresponding residues across various homologous bacterial RNase III proteins, or homologs.

Mutations in the RNaseIII refer to any of point mutations, additions, deletions (though preferably not in the cleavage domain), and rearrangements (preferably in the domain linking regions). Mutations may be at a single site or at multiple sites in the RNaseIII protein. Mutations can be generated by standard techniques including random mutagenesis, targeted genetics and other methods know by those of ordinary skill in the art.

A further aspect of the invention relates to the use of DNA editing compositions and methods to inhibit, alter, disrupt expression, and/or replace one or more target genes, for example through homologous recombination. In various embodiments, one or more target genes may be altered through CRISPR/Cas-9, TALAN, or Zinc (Zn2+) finger nuclease systems. In some embodiments, the agent for altering gene expression is CRISPR-Cas9, or a functional equivalent thereof, together with an appropriate RNA molecule arranged to target one or more target genes, such as RNaseIII or any homolog/orthologs thereof. For example, one embodiment of the present invention may include the introduction of one or more guide RNAs (gRNAs) to be utilized by CRISPR/Cas9 system to disrupt, replace, or alter the expression or activity of one or more target genes.

In this context, the gene-editing CRISPR/cas-9 technology is an RNA-guided gene-editing platform that makes use of a bacterially derived protein (Cas9) and a synthetic guide RNA to introduce a double strand break at a specific location within the genome. Editing is achieved by transfecting a cell or a subject with the Cas9 protein along with a specially designed guide RNA (gRNA) that directs the cut through hybridization with its matching genomic sequence. By making use of this technology, it is possible to introduce specific genetic alterations in one or more target genes. In some embodiments, this CRISPR/cas-9 may be utilized to replace one or more existing wild-type genes with a modified version, while additional embodiments may include the addition of genetic elements that alter, reduce, increase or knock-out the expression of a target gene such as endogenous RNaseIII in Csr-7 or Csr-8.

In some embodiments, the agent for altering gene expression is a zinc finger, or zinc finger nuclease or other equivalent. The term “zinc finger nuclease” or “zinc finger nuclease as used herein, refers to a nuclease comprising a nucleic acid cleavage domain conjugated to a binding domain that comprises a zinc finger array. In some embodiments, the cleavage domain is the cleavage domain of the type II restriction endonuclease Fokl. Zinc finger nucleases can be designed to target virtually any desired sequence in a given nucleic acid molecule for cleavage, and the possibility to design zinc finger binding domains to bind unique sites in the context of complex genomes allows for targeted cleavage of a single genomic site in living cells, for example, to achieve a targeted genomic alteration of therapeutic value. Targeting a double-strand break to a desired genomic locus can be used to introduce frame-shift mutations into the coding sequence of a gene due to the error-prone nature of the non-homologous DNA repair pathway. Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich NP, Pabo Colo. (May 1991). “Zinc finger-DNA recognition: crystal structure of a Zif268-DNA complex at 2.1 A”. Science 252 (5007): 809-17, the entire contents of which are incorporated herein). In some embodiments, separate zinc fingers that each recognizes a 3 base pair DNA sequence are combined to generate 3-, 4-, 5-, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4-, 6-, or 8-zinc finger arrays. In some embodiments, bacterial or phage display is employed to develop a zinc finger domain that recognizes a desired nucleic acid sequence, for example, a desired nuclease target site of 3-30 bp in length.

Zinc finger nucleases, in some embodiments, comprise a zinc finger binding domain and a cleavage domain fused or otherwise conjugated to each other via a linker, for example, a polypeptide linker. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a Fokl cleavage domain, and one monomer comprising zinc finger domain B conjugated to a Fokl cleavage domain. In this nonlimiting example, zinc finger domain A binds a nucleic acid sequence on one side of the target site, zinc finger domain B binds a nucleic acid sequence on the other side of the target site, and the dimerize Fokl domain cuts the nucleic acid in between the zinc finger domain binding sites.

The term “zinc finger,” as used herein, refers to a small nucleic acid-binding protein structural motif characterized by a fold and the coordination of one or more zinc ions that stabilize the fold. Zinc fingers encompass a wide variety of differing protein structures (see, e.g., Klug A, Rhodes D (1987). “Zinc fingers: a novel protein fold for nucleic acid recognition”. Cold Spring Harb. Symp. Quant. Biol. 52: 473-82, the entire contents of which are incorporated herein by reference). Zinc fingers can be designed to bind a specific sequence of nucleotides, and zinc finger arrays comprising fusions of a series of zinc fingers, can be designed to bind virtually any desired target sequence. Such zinc finger arrays can form a binding domain of a protein, for example, of a nuclease, e.g., if conjugated to a nucleic acid cleavage domain. Different types of zinc finger motifs are known to those of skill in the art, including, but not limited to, Cys2His2, Gag knuckle, Treble clef, Zinc ribbon, Zn2/Cys6, and TAZ2 domain-like motifs (see, e.g., Krishna S, Majumdar I, Grishin N V (January 2003). “Structural classification of zinc fingers: survey and summary”. Nucleic Acids Res. 31 (2): 532-50). Typically, a single zinc finger motif binds 3 or 4 nucleotides of a nucleic acid molecule. Accordingly, a zinc finger domain comprising 2 zinc finger motifs may bind 6-8 nucleotides, a zinc finger domain comprising 3 zinc finger motifs may bind 9-12 nucleotides, a zinc finger domain comprising 4 zinc finger motifs may bind 12-16 nucleotides, and so forth. Any suitable protein engineering technique can be employed to alter the DNA-binding specificity of zinc fingers and/or design novel zinc finger fusions to bind virtually any desired target sequence from 3-30 nucleotides in length (see, e.g., Pabo C O, Peisach E, Grant RA (2001). “Design and selection of novel cys2H is2 Zinc finger proteins”. Annual Review of Biochemistry 70: 313-340; Jamieson A C, Miller J C, Pabo C O (2003). “Drug discovery with engineered zinc-finger proteins”. Nature Reviews Drug Discovery 2 (5): 361-368; and Liu Q, Segal D J, Ghiara J B, Barbas C F (May 1997). “Design of polydactyl zinc-finger proteins for unique addressing within complex genomes”. Proc. Natl. Acad. Sci. U.S.A. 94 (11); the entire contents of each of which are incorporated herein by reference).

Fusions between engineered zinc finger arrays and protein domains that cleave a nucleic acid can be used to generate a “zinc finger nuclease.” A zinc finger nuclease typically comprises a zinc finger domain that binds a specific target site within a nucleic acid molecule, and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Longer target sites are particularly attractive in situations where it is desired to bind and cleave a target site that is unique in a given genome.

In some embodiments, the agent for altering the target gene is a TALEN system or its equivalent. The term TALEN or “Transcriptional Activator-Like Element Nuclease” or “TALE nuclease” as used herein, refers to an artificial nuclease comprising a transcriptional activator like effector DNA binding domain to a DNA cleavage domain, for example, a Fokl domain. A number of modular assembly schemes for generating engineered TALE constructs have been reported (Zhang, Feng; et. al. (February 2011). “Efficient construction of sequence-specific TAL effectors for modulating mammalian transcription”. Nature Biotechnology 29 (2): 149-53; Geibler, R.; Scholze, H.; Hahn, S.; Streubel, J.; Bonas, U.; Behrens, S. E.; Boch, J. (2011), Shiu, Shin-Han. ed. “Transcriptional Activators of Human Genes with Programmable DNA- Specificity”. PLoS ONE 6 (5): el9509; Cermak, T.; Doyle, E. L.; Christian, M.; Wang, L.; Zhang, Y.; Schmidt, C; Bailer, J. A.; Somia, N. V. et al. (2011). “Efficient design and assembly of custom TALEN and other TAL effector-based constructs for DNA targeting”. Nucleic Acids Research; Morbitzer, R.; Elsaesser, J.; Hausner, J.; Lahaye, T. (2011). “Assembly of custom TALE-type DNA binding domains by modular cloning”. Nucleic Acids Research; Li, T.; Huang, S.; Zhao, X.; Wright, D. A.; Carpenter, S.; Spalding, M. H.; Weeks, D. P.; Yang, B. (2011). “Modularly assembled designer TAL effector nucleases for targeted gene knockout and gene replacement in eukaryotes”. Nucleic Acids Research.; Weber, E.; Gruetzner, R.; Werner, S.; Engler, C; Marillonnet, S. (2011). Bendahmane, Mohammed, ed. “Assembly of Designer TAL Effectors by Golden Gate Cloning”. PLoS ONE 6 (5): el9722; each of which is incorporated herein by reference). Those of skill in the art will understand that TALE nucleases can be engineered to target virtually any genomic sequence with high specificity, and that such engineered nucleases can be used in embodiments of the present technology to manipulate the genome of a cell, e.g., by delivering the respective TALEN via a method or strategy disclosed herein under circumstances suitable for the TALEN to bind and cleave its target sequence within the genome of the cell. In some embodiments, the delivered TALEN targets a gene, such as an endogenous RNaseIII.

In some embodiments, the target gene of a cell, tissue, organ, or organism is altered by a nuclease delivered to the cell via a strategy or method disclosed herein, e.g., CRISPR/cas-9, a TALEN, or a zinc-finger nuclease, or a plurality or combination of such nucleases. In some embodiments, a single- or double-strand break is introduced at a specific site within the genome by the nuclease, resulting in a disruption of the target genomic sequence.

In some embodiments, the target genomic sequence is a nucleic acid sequence within the coding region of a target gene. In some embodiments, the strand break introduced by the nuclease leads to a mutation within the target gene that impairs the expression of the encoded gene product. In some embodiments, a nucleic acid is co-delivered to the cell with the nuclease. In some embodiments, the nucleic acid comprises a sequence that is identical or homologous to a sequence adjacent to the nuclease target site. In some such embodiments, the strand break affected by the nuclease is repaired by the cellular DNA repair machinery to introduce all or part of the co-delivered nucleic acid into the cellular DNA at the break site, resulting in a targeted insertion of the co-delivered nucleic acid, or part thereof. In some embodiments, the insertion results in the disruption or repair of the undesired allele. In some embodiments, the nucleic acid is co-delivered by association to a supercharged protein. In some embodiments, the supercharged protein is also associated to the functional effector protein, e.g., the nuclease. In some embodiments, the delivery of a nuclease to a target cell results in a clinically or therapeutically beneficial alteration of the function of a gene.

In some embodiments, cells from a subject are obtained and a nuclease or other effector protein is delivered to the cells by a system or method provided herein ex vivo. In some embodiments, the treated cells are selected for those cells in which a desired nuclease-mediated genomic editing event has been affected. In some embodiments, treated cells carrying a desired genomic mutation or alteration are returned to the subject they were obtained from.

According to the present invention “sRNA” is small RNA, in particular RNA of a length of 200 nucleotides or less that is not translated into a protein. sRNA may be an RNA molecules digested by one or more of the RNaseIII mutants described herein. sRNA may include siRNA mRNA, or even dsRNA molecules that may be generated by or initiate an RNAi pathway response which may result in the downregulation of a target gene. “RNAi” refers to gene downregulation or inhibition that is induced by the introduction of a double-stranded RNA molecule.

As used herein, RNA interference (RNAi) is a biological mechanism which leads to post transcriptional gene silencing (PTGS) triggered by double-stranded RNA (dsRNA) molecules, for example provided by hpRNA, to prevent the expression of specific genes. For example, RNA interference may be accomplished as short hpRNA molecules may be imported directly into the cytoplasm, anneal together to form a dsRNA, and then cleaved to short fragments by the DICER enzyme. This enzyme DICER may process the dsRNA into -21 - 22-nucleotide fragment with a 2 -nucleotide overhang at the 3′ end, small interfering RNAs (siRNAs). The antisense strand of siRNA become specific to endonuclease-protein complex, RNA-induced silencing complex (RISC), which then targets the homologous RNA and degrades it at specific site that results in the knock-down of protein expression. Endophytic bacteria that may transmit hpRNA, dsRNA, shRNA, siRNA, and microRNA species to plants. In one preferred embodiment, MM-W1 and/or MM-W2 may infect or otherwise colonize a target host, and may be transformed with artificially created genetic constructs, such as plasmids or chromosomal integration, that may generate the inhibitory RNA molecules. In this preferred embodiment, one or more select inhibitory RNA molecules, and preferably hpRNA molecules directed to one or more essential genes of a pathogen, such as WSSV, which may be expressed in a probiotic enteric bacteria such as MM-W1 and/or MM-W2, that may further include an engineered E. coli RNaseIII mutant E38A/R107A/R108A gene configured to generate siRNAs in a DICER-independent pathway the bacteria was added to the hpRNA expression constructs - which may further be coupled with a rnc knockout as generally described herein. In this embodiment, the hpRNA are processed into siRNA molecules that may be delivered to a target, which in a preferred embodiment may be a shrimp, and down-regulate or eliminate replication and/or translation of proteins of one or more pathogens, such as WSSV.

In still other embodiments of the invention, inhibition of the expression of one or more pathogen gene products by RNAi may be obtained through a dsRNA-mediated RNAi action and/or a form of dsRNA known as a hairpin RNA (hpRNA) interference or intron-containing hairpin RNA (ihpRNA) interference. For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene product whose expression is to be inhibited, in this case, a pathogen essential gene described herein, and an antisense sequence that is fully or partially complementary to the sense sequence. Alternatively, the base-paired stem region may correspond to a portion of a promoter sequence controlling expression of the gene encoding the target polypeptide to be inhibited. Thus, the base-paired stem region of the molecule generally determines the specificity of the RNA interference. HpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97:4985-4990; Stoutjesdijk et al. (2002) Plant Physiol. 129: 1723-1731; and Waterhouse and Helliwell (2003) Nat. Rev. Genet. 4:29-38. Methods for using hpRNA interference to inhibit or silence the expression of genes are described, for example, in Chuang and Meyerowitz (2000) Proc. Natl. Acad. Sci. USA 97:4985-4990; Stoutjesdijk et al. (2002) Plant Physiol. 129: 1723-1731; Waterhouse and Helliwell (2003) Nat. Rev. Genet. 4:29-38; Pandolfmi et al. BMC Biotechnology 3:7, and U.S. Pat. Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 407:319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 407:319-320; Wesley et al. (2001) Plant J 27:581-590; Wang and Waterhouse (2001) Curr. Opin. Plant Biol. 5: 146-150; Waterhouse and Helliwell (2003) Nat. Rev. Genet. 4:29-38; Helliwell and Waterhouse (2003) Methods 30:289-295, and U.S. Pat. Publication No. 20030180945, each of which is herein incorporated by reference.

The term “gene” or “gene sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (e.g., introns) between individual coding regions (e.g., exons). The term “structural gene” as used herein is intended to mean a DNA sequence that is transcribed into mRNA which is then translated into a sequence of amino acids characteristic of a specific polypeptide.

As used herein, “inhibit”, “inhibition,” “suppress,” “downregulate” or “silencing” refers to partial or complete loss-of-function through targeted inhibition of gene expression in a cell and may also be referred to as “knock down,” preferably through an RNAi pathway response. Depending on the circumstances and the biological problem to be addressed, it may be preferable to partially reduce gene expression. Alternatively, it might be desirable to reduce gene expression as much as possible. The extent of silencing may be determined by any method known in the art, some of which are summarized in International Publication No. WO99/32619, incorporated herein by reference. As used herein, “inhibit”, “inhibition”, “suppress,” “downregulate,” or “silencing” of the level or activity of an agent, such as, for example, a preRNA, mRNA, rRNA, tRNA, snoRNA, snRNA expressed by the target gene, and/or of the protein product encoded by it, means that the amount is reduced by 10% or more, for example, 20% or more, preferably 30% or more, more preferably 50% or more, even more preferably 70% or more, most preferably 80% or more, for example, 90%, relative to a cell or organism lacking a dsRNA molecule of the disclosure.

“Expression” or “expressing” refers to production of a functional product, such as, the generation of an RNA transcript from an introduced construct, an endogenous DNA sequence, or a stably incorporated heterologous DNA sequence. A nucleotide encoding sequence may comprise intervening sequence (e.g., introns) or may lack such intervening non-translated sequences (e.g., as in cDNA). Expressed genes include those that are transcribed into mRNA and then translated into protein and those that are transcribed into RNA but not translated (for example, siRNA, transfer RNA, and ribosomal RNA). The term may also refer to a polypeptide produced from an mRNA generated from any of the above DNA precursors. Thus, expression of a nucleic acid fragment, such as a gene or a promoter region of a gene, may refer to transcription of the nucleic acid fragment (e.g., transcription resulting in mRNA or other functional RNA) and/or translation of RNA into a precursor or mature protein (polypeptide), or both.

An “expression cassette” or “expression vector” or “vector” refers to a nucleic acid construct, which when introduced into a host cell, results in transcription and/or translation of an RNA or polypeptide, respectively. More specifically, the term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria. Again, more specifically, “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). A polynucleotide sequence is operably linked to an expression control sequence(s) (e.g., a promoter and, optionally, an enhancer) when the expression control sequence controls and regulates the transcription and/or translation of that polynucleotide sequence.

The term “genome” encompasses not only chromosomal DNA found within the nucleus, but organelle DNA found within subcellular components (e.g., mitochondrial, plastid) of the cell. As used herein, the term “genome” refers to the nuclear genome unless indicated otherwise.

As used herein, the term “eukaryotic-like RNA” or “eukaryotic-like mRNA” refers to an RNA molecule expressed in a prokaryotic or other non-eukaryotic systems that is competent to be expressed in a recipient eukaryotic cell.

Notably, all DNA sequences provided may encompass all RNA and amino acid sequences, and vice versa as would be ascertainable by those of ordinary skill in the art, for example through Uracil substitutions as well as redundant codons. Additionally, all sequences include codon-optimized embodiments as would be ascertainable by those of ordinary skill in the art. As such, the term “encoding” or “coding sequence” or “coding” means both encoding a nucleotide and/or amino acid sequence and vice versa.

The term “heterologous” refers to a nucleic acid fragment or protein that is foreign to its surroundings. In the context of a nucleic acid fragment, this is typically accomplished by introducing such fragment, derived from one source, into a different host. Heterologous nucleic acid fragments, such as coding sequences that have been inserted into a host organism, are not normally found in the genetic complement of the host organism. As used herein, the term “heterologous” also refers to a nucleic acid fragment derived from the same organism, but which is located in a different, e.g., non-native, location within the genome of this organism. Thus, the organism can have more than the usual number of copy(ies) of such fragment located in its(their) normal position within the genome and in addition, in the case of plant cells, within different genomes within a cell, for example in the nuclear genome and within a plastid or mitochondrial genome as well. A nucleic acid fragment that is heterologous with respect to an organism into which it has been inserted or transferred is sometimes referred to as a “transgene.”

“Host cell” means a cell which contains an expression vector and supports the replication and/or expression of that vector. The term “introduced” means providing a nucleic acid (e.g., an expression construct) or protein into a cell. “Introduced” includes reference to the incorporation of a nucleic acid into a eukaryotic or prokaryotic cell where the nucleic acid may be incorporated into the genome of the cell and includes reference to the transient provision of a nucleic acid or protein to the cell. “Introduced” includes reference to stable or transient transformation methods, as well as sexually crossing. Thus, “introduced” in the context of inserting a nucleic acid fragment ( e.g., a recombinant DNA construct/ expression construct) into a cell, can mean “transfection” or “transformation” or “transduction”, and includes reference to the incorporation of a nucleic acid fragment into a eukaryotic or prokaryotic cell where the nucleic acid fragment may be incorporated into the genome of the cell ( e.g., chromosome, plasmid, plastid, or mitochondrial DNA), converted into an autonomous replicon, or transiently expressed (e.g., transfected mRNA).

As used herein, “nucleic acid” or “nucleotide sequence” means a polynucleotide ( or oligonucleotide), including single or double-stranded polymers of deoxyribonucleotides or ribonucleotide bases, and unless otherwise indicated, encompasses naturally occurring and synthetic nucleotide analogues having the essential nature of natural nucleotides in that they hybridize to complementary single stranded nucleic acids in a manner similar to naturally occurring nucleotides. Nucleic acids may also include fragments and modified nucleotide sequences. Nucleic acids disclosed herein can either be naturally occurring, for example genomic nucleic acids, or isolated, purified, nongenomic nucleic acids, including synthetically produced nucleic acid sequences such as those made by solid phase chemical oligonucleotide synthesis, enzymatic synthesis, or by recombinant methods, including for example, cDNA, codon- optimized sequences for efficient expression in different transgenic plants reflecting the pattern of codon usage in such plants, nucleotide sequences that differ from the nucleotide sequences disclosed herein due to the degeneracy of the genetic code but that still encode the protein(s) of interest disclosed herein, nucleotide sequences encoding the presently disclosed protein(s) comprising conservative (or non-conservative) amino acid substitutions that do not adversely affect their normal activity, PCR-amplified nucleotide sequences, and other non-genomic forms of nucleotide sequences familiar to those of ordinary skill in the art.

The term “sequence identity” or “identity,” as used herein in the context of two nucleic acid or polypeptide sequences, refers to the residues in the two sequences that are the same when aligned for maximum correspondence over a specified comparison window.

“Operably linked” refers to a functional arrangement of elements. A first nucleic acid sequence is operably linked with a second nucleic acid sequence when the first nucleic acid sequence is placed in a functional relationship with the second nucleic acid sequence. For instance, a promoter is operably linked to a coding sequence if the promoter effects the transcription or expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

The terms “peptide”, “polypeptide”, and “protein” are used to refer to polymers of amino acid residues. These terms are specifically intended to cover naturally occurring biomolecules, as well as those that are recombinantly or synthetically produced, for example by solid phase synthesis.

The term “promoter” or “regulatory element” refers to a region or nucleic acid sequence located upstream or downstream from the start of transcription and which is involved in recognition and binding of RNA polymerase and/or other proteins to initiate transcription of RNA.

As used herein, the term “transformation” or “genetically modified” refers to the transfer of one or more nucleic acid molecule(s) into a cell. A microorganism is “transformed” or “genetically modified” by a nucleic acid molecule transduced into the bacteria when the nucleic acid molecule becomes stably replicated by the bacteria. As used herein, the term “transformation” or “genetically modified” encompasses all techniques by which a nucleic acid molecule can be introduced into, such as a bacterium.

“Stable transformation” or “integration” or “stable integration” is intended to mean that the nucleotide construct introduced into a host and integrates into the genome of the plant and is capable of being inherited by the progeny thereof. The nucleic acid molecule can be transiently expressed or non-stably maintained in a functional form in the cell for less than three months e.g. is transiently expressed.

The term “prokaryotic” is meant to include all bacteria, archaea, and/or cyanobacteria which can be transformed or transfected with a nucleic acid and express a eukaryotic-like RNA of the invention. Prokaryotic hosts may include gram negative as well as gram positive bacteria. The term “eukaryotic” is meant to include yeast, algae, plants, higher plants, insect, and mammalian cells.

“Target” or “essential gene” refers to any gene or mRNA of interest. Indeed, any of the genes previously identified by genetics or by sequencing may represent a target. Target genes or mRNA may include developmental genes and regulatory genes as well as metabolic or structural genes or genes encoding enzymes. The target gene may be expressed in those cells in which a phenotype is being investigated or in an organism in a manner that directly or indirectly impacts a phenotypic characteristic. The target gene may be endogenous or exogenous. An “essential gene,” for example may be a gene necessary for survival, replication, or pathogenicity in a pathogen such as, for example WSSV.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as is known to one of ordinary skill in the art and is understood as included in embodiments where it would be appropriate. Nucleotides may be referred to by their commonly accepted single-letter codes. Unless otherwise indicated, amino acid sequences are written left to right in amino to carboxyl orientation, respectively. Amino acids may be referred to herein by either their commonly known three letter symbols or by the one-letter symbols as generally understood by those skilled in the relevant art.

Notably, all peptides disclosed in specifically encompass peptides having conservative amino acid substitutions. As used herein, “conservative amino acid substitutions” means the manifestation that certain amino acids can be substituted for other amino acids in a protein structure without appreciable loss of biochemical or biological activity. Since it is the interactive capacity and nature of a protein that defines that protein’s biological functional activity, certain amino acid sequence substitutions can be made in a protein sequence, and, of course, the underlying DNA coding sequence, and nevertheless obtain a protein with like properties. Thus, various changes can be made in the amino acid sequences disclosed herein, or in the corresponding DNA sequences that encode these amino acid sequences, without appreciable loss of their biological utility or activity.

Examples of amino acid groups defined in this manner include: a “charged polar group,” consisting of glutamic acid (Glu), aspartic acid (Asp), asparagine (Asn), glutamine (Gln), lysine (Lys), arginine (Arg) and histidine (His); an “aromatic, or cyclic group,” consisting of proline (Pro), phenylalanine (Phe), tyrosine (Tyr) and tryptophan (Trp); and an “aliphatic group” consisting of glycine (Gly), alanine (Ala), valine (Val), leucine (Leu), isoleucine (Ile), methionine (Met), serine (Ser), threonine (Thr) and cysteine (Cys).

Within each group, subgroups can also be identified, for example, the group of charged polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gin. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala. Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gin for Asn such that a free —NH2 can be maintained.

Proteins and peptides biologically functionally equivalent to the proteins and peptides disclosed herein include amino acid sequences containing conservative amino acid changes in the fundamental amino acid sequence. In such amino acid sequences, one or more amino acids in the fundamental sequence can be substituted, for example, with another amino acid(s), the charge and polarity of which is similar to that of the native amino acid, i.e., a conservative amino acid substitution, resulting in a silent change. It should be noted that there are a number of different classification systems in the art that have been developed to describe the interchangeability of amino acids for one another within peptides, polypeptides, and proteins. The following discussion is merely illustrative of some of these systems, and the present disclosure encompasses any of the “conservative” amino acid changes that would be apparent to one of ordinary skill in the art of peptide, polypeptide, and protein chemistry from any of these different systems. Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. Table 13, infra, contains information about which nucleic acid codons encode which amino acids.

TABLE 13

Amino acid Nucleic acid codons

| | |
|---|---|
| Amino Acid | Nucleic Acid Codons |
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

As used herein, the term “eukaryotic-like RNA” or “eukaryotic-like mRNA” refers to an RNA molecule expressed in a prokaryotic or other non-eukaryotic systems that is competent to be expressed in a recipient eukaryotic cell.

Notably, all DNA sequences provided may encompass all RNA and amino acid sequences, and vice versa as would be ascertainable by those of ordinary skill in the art, for example through Uracil substitutions as well as redundant codons. Additionally, all sequences include codon-optimized embodiments as would be ascertainable by those of ordinary skill in the art. As such, the term “encoding” or “coding sequence” or “coding” means both encoding a nucleotide and/or amino acid sequence and vice versa.

As used herein “auxotroph” or “auxotrophic” refers to a microorganism having a specific nutritional requirement NOT required by the wild-type organism. In the absence of the required nutrient the auxotroph will not grow whereas the wild-type will thrive.

In a further embodiment, a composition including a genetically modified bacteria configured to express one or more RNase III mutants that produce sRNA may be formulated as feed, such as an animal feed, and/or a water dispersible granule or powder that may further be configured to be dispersed into the environment. In yet a further embodiment, the compositions of the present invention may also comprise a wettable powder, spray, emulsion, colloid, aqueous or organic solution, dust, pellet, or colloidal concentrate. Dry forms of the compositions may be formulated to dissolve immediately upon wetting, or alternatively, dissolve in a controlled-release, sustained- release, or other time-dependent manner. Alternatively, or additionally, the composition may comprise an aqueous solution. Such aqueous solutions or suspensions may be provided as a concentrated stock solution which is diluted prior to application, or alternatively, as a diluted solution ready -to-apply. Such compositions may be formulated in a variety of ways. They may be employed as wettable powders, granules, or dusts, by mixing with various inert materials, such as inorganic minerals (silicone or silicon derivatives, phyllosilicates, carbonates, sulfates, phosphates, and the like) or botanical materials (powdered corncobs, rice hulls, walnut shells, and the like). The formulations or compositions containing genetically modified bacteria may include spreader- sticker adjuvants, stabilizing agents, other pesticidal additives, or surfactants. Liquid formulations may be employed as foams, suspensions, emulsifiable concentrates, or the like. The ingredients may include Theological agents, surfactants, emulsifiers, dispersants, or polymers. According to one embodiment, the composition is administered to the host by feeding. Feeding the host with the composition can be effected once, regularly, or semi-regularly over the span of hours, days, weeks, months or even years.

As mentioned, the sRNA of the invention may be administered as a naked sRNA. Alternatively, the sRNA of the invention may be conjugated to a carrier known to one of skill in the art, such as a transfection agent e.g. PEI or chitosan or a protein/ lipid carrier or coupled to nanoparticles. The compositions may be formulated prior to administration in an appropriate means such as lyophilized, freeze-dried, microencapsulated, desiccated, or in an aqueous carrier, medium or suitable diluent, such as saline or another buffer. Suitable agricultural carriers can be solid, semi-solid or liquid and are well known in the art. Such compositions may be considered “acceptable carriers”, which may cover all adjuvants, e.g., inert components, dispersants, surfactants, tackifiers, binders, etc.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. As used herein, the term “treating” includes abrogating, substantially inhibiting, slowing, or reversing the progression of a condition, substantially ameliorating clinical or aesthetical symptoms of a condition, or substantially preventing the appearance of clinical or aesthetical symptoms of a condition.

According to some embodiments, the nucleic acid agent is provided in amounts effective to reduce or suppress expression of at least one host pathogen resistance gene product. As used herein “a suppressive amount” or “an effective amount” or a “therapeutically effective amount” refers to an amount of dsRNA which is sufficient to downregulate (reduce expression of) the target gene by at least 20%, 30%, 40%, 50%, or more, say 60%, 70%, 80%, 90% or more even 100%, or reduce mortality in an animal or animal population, such as shrimp in aquaculture by at least a measurable percentage, preferably between 1%-100%.

This invention utilizes routine techniques in the field of molecular biology. Basic texts disclosing the general methods of use in this invention include Green and Sambrook, 4th ed. 2012, Cold Spring Harbor Laboratory; Kriegler, Gene Transfer and Expression: A Laboratory Manual (1993); and Ausubel et al., eds., Current Protocols in Molecular Biology, 1994-current, John Wiley & Sons. Unless otherwise noted, technical terms are used according to conventional usage. Definitions of common terms in molecular biology maybe found in e.g., Benjamin Lewin, Genes IX, published by Oxford University Press, 2007 (ISBN 0763740632); Krebs, et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Overall Scheme of Gene Integrations and Knockouts

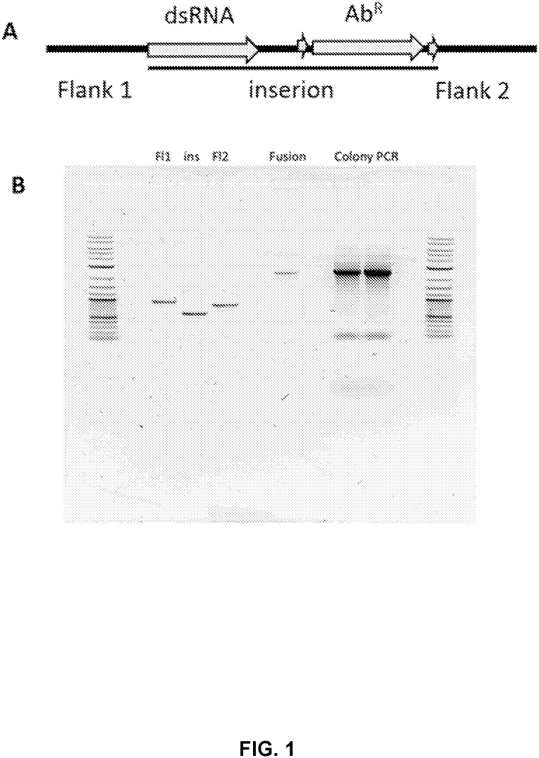

Gene integrations and knockouts were performed by double-crossover recombination into Bacillus genome to generate MM-W1 and MM-W2 as described in Table 1. The present inventors used fusion PCR to create long DNA fragments that consists 5′ and 3′ flanks that were homologous to the integration sites. Between these flanks were target expression cassettes (optional for integration) and an antibiotic gene overlapped by lox sequences that allow antibiotic markers removal. A recombination event removed sequences at the integration site and exchange them to the sequences in the corresponding DNA fragment. Selection was based on the antibacterial activity of corresponding antibiotic marker. The general scheme for the generation of MM-W1 and MM-W2 is presented on FIG. 1.

As antibiotic marker, the present inventors used zeocin resistance marker that produces protein that binds and sequester zeocin thus rendering Bacillus zeocin resistant. Source of zeocin marker overlapped with lox71 and lox66 was engineered into plasmid p7Z6 (See Table 1). Using this fragment, zeocin marker was PCR amplified to create gene integrations/knockout constructs. To create GM Bacillus strains that grow as efficiently as wild type we selected two biologically neutral sites. First, chloramphenicol resistance marker located in B. Subtilis Δ6 genome instead of the polyketide synthase operon. Knockout of this artificial marker and replacement it with our construct would not lead to growth defect. A second integration site was chosen to include the ywd operon. The Ywd operon function is unknown, but neutrality of this site was verified by two independent knockouts. It was shown that ywdD gene-knockout does not affect Bacillus growth at any of growth conditions tested.

### Example 2: Antibiotic Marker Removal After Genomic Integration

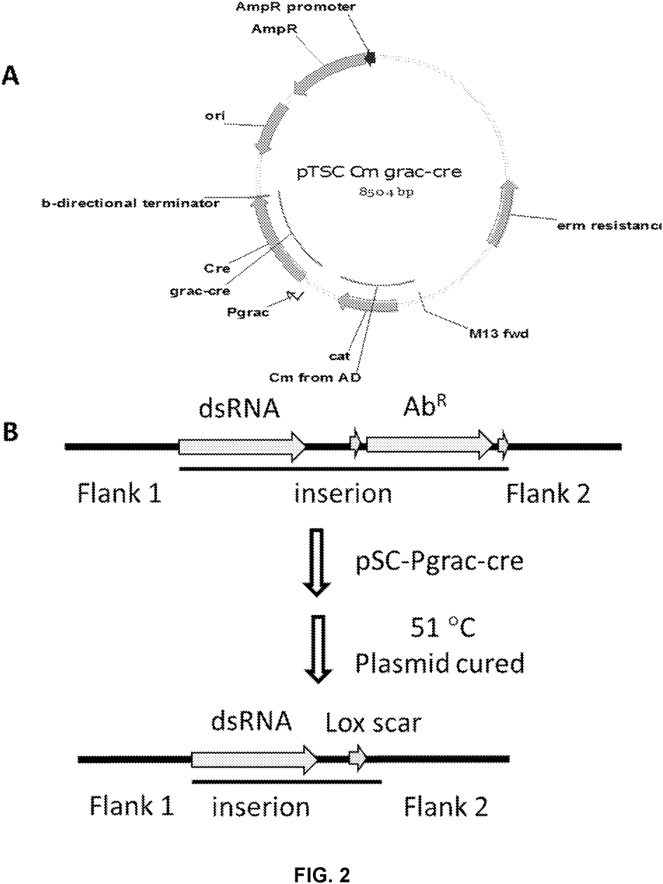

Removal of antibiotic markers from Bacillus genome was required in order to perform consequent genome modification events and to produce a final marker-less bacteria that are safe to environment. To facilitate the antibiotic marker removal, the present inventors employed Crelox system: antibiotic markers were overlapped by lox markers that could be recognized and removed by Cre recombinase. The present inventors expressed Cre recombinase in Bacillus using the shuttle vector that could replicate in both E.coli and Bacillus, and the plasmid replication in Bacillus is thermo-sensitive at temperatures above 51° C. Freely available plasmid pTSC contains Cre gene without any promoter and ribosome binding site, therefore, antibiotic marker removal was very inefficient. As a result, the present inventors modified this plasmid by introducing chloramphenicol resistance marker and by replacing promoter-less Cre gene with Pgrac-cre cassette that is optimized for expression (see FIGS. 2A-B). Efficiency of marker removal with pSC-Pgrac-cre was almost 100% that significantly shortened time of experiments.

### Example 3: Comparison of VP19 Expression Directed by Pupp and Pgrac Promoters

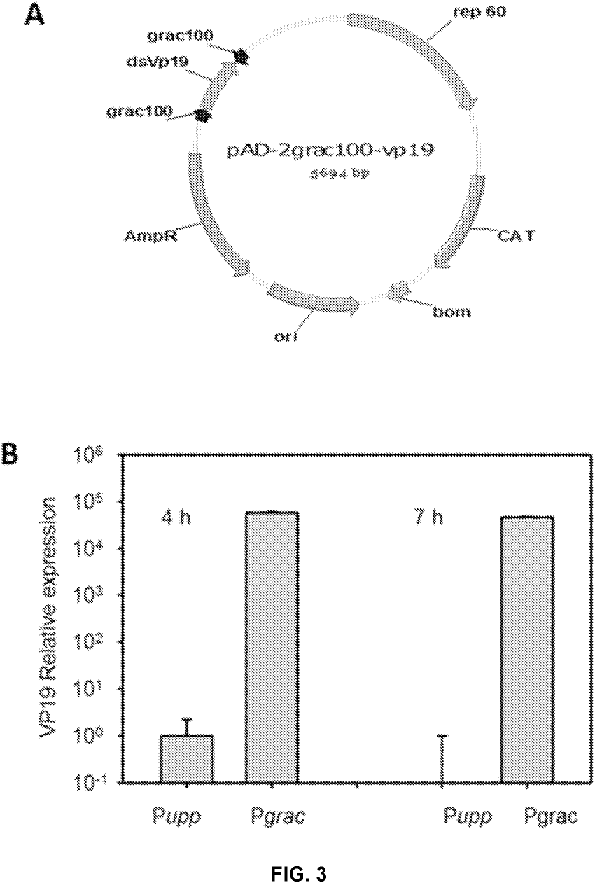

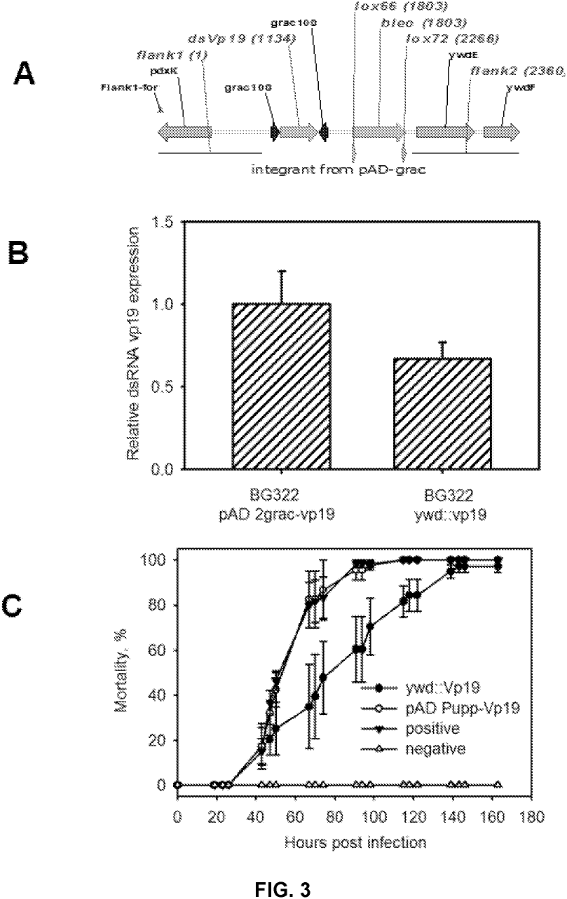

Nucleotide constructs expressing dsRNA selected from SEQ ID NOs. 1-3, were operably linked to the strong promoter of Bacillus cereus upp gene. In order to optimize expression, the present inventors replaced the upp promoter with an artificial Pgrac100 promoter. (See FIG. 3 for description of plasmid pAD-2grac100-vp19 via Genescript) Plasmid pAD-2grac100-vp19 was transformed into the Δrnc Bacillus strain BG322. Both strains BG322 (pAD 2grac100-vp19) and BG322 (pAD Pupp-vp19) were grown in LB and aliquots were taken at certain time points to analyze dsRNA-vp19 expression by qRT-PCR. As shown in FIG. 3, production of dsRNA-vp19 is 10,000 folds higher when expression is directed by Pgrac promoter. Thus, the present inventors concluded that antiviral constructs using Pgrac promoter would be superior compared to prior traditional promoter constructs.

### Example 4: Stable Integration of dsRNA-vp19 Expressing Cassette Leads to the Better Shrimp protection against WSSV.

As shown in FIG. 4, the present inventors compared anti-viral properties of plasmid based dsRNA vp19 construct and stably integrated line (A). While integrated line demonstrates slightly lower but similar expression of dsRNA-vp19 (B), it is superior in decreasing shrimp mortality in WSSV infection. (C). Thus, the present inventors concluded that integrative lines would be superior compared with plasmid based constructs we described by Sayre et al. in PCT/US2018/025766.

### Example 5: Characterization of RNAseIII-N3XT Activity in Bacillus Subtilis Strain 168δ6

E. coli RNAseIII-N3XT (selected from SEQ ID NO. 4-13) function in Bacillus 168Δ6 was evaluated by Northern blot hybridization in strains expressing dsRNA:Vp19 (WSSV targeting). As shown in FIG. 5, the present inventors observed that: RNAseIII-N3XT activity in Bacillus 168Δ6:Δrnc leads to accumulation of smRNA duplexes of 21-23nt and also in the 26nt and 30nt range; 2) Δrnc mutation leads to higher accumulation of long dsRNA precursors; and 3) RNAse-N3XT is apparently unable to compete with WT rnc as no smRNA accumulation was observed when both enzymes are present.

Thus, we conclude that in MM-W1, E coli RNAse-N3XT activity results in accumulation of RISC competent vp19 smRNA duplexes (21-23nt) in Bacillus 168Δ6.

### Example 6: MM-W1 and MM-W2 Viability

To compare viability of strains MM-W1 and MM-W2 the present inventors compared bacterial growth of wt and genetically engineering Bacillus strains at 37° C. in LB, starting OD <0.05, 250 rpm. As shown in Table 2 below, doubling times are very similar, demonstrating that MM-W1 and MM-W2 grow as well as wt Bacillus strains.

### TABLES

TABLE 1

Strain genotypes

| Strain name | Description | Source |
|---|---|---|
| Bacillus subtilis | | |
| Δ6 | Cm, trpC2, Δskin, ICEBs1(0), Δprophage1, Δprophage3, ΔSPbeta, ΔPBSX, pks::Cm | Bacillus Genetic Stock Center (1A1299) |
| MM-W0 | Δ6 cat::lox | Present invention |
| MM-W1 | Δ6 cat::Pgrac-VP19 rnc::lox ywdD::N3XT | Present invention |
| MM-W2 | Δ6 cat::Pgrac-VP19 rnc::lox | Present invention |
| MM-W3 | Δ6 cat::Pgrac-VP19 ywdD::N3XT Where N3XT is E.coli E38A-R107A-R108A RNaseIII under Pveg promoter | Present invention |
| MM-W4 | Δ6 cat::lox rnc::lox | Present invention |
| MM-W0 Δrnc (pAD 2grac100-vp19) | Cm^{R}, contain vp19 expressing transcriptional units under Pgrac bidirectional promoter | transformed in MM-W4 |
| BG322 | | |
| BG322 | ywdD::Pgrac-Vp19 | Present invention |
| BG322 (pAD 2grac100-vp19) | | Present invention |
| Plasmids | Description | Source |
| pAD-2grac100-vp19 | pAD43-25 E. coli/Bacillus subtilis shuttle vector where dsRNA Vp19 is expressed under bidirectional Pgrac promoter | Present invention, Vp19 cassette made by GENSCRIPT |
| pAD-WRKY-GHY7-E38A-R107A-R108A | pAD43-25 E. coli/B. subtilis shuttle vector where E. coli RNaseIII (E38A-R107A-R108A) expressed under control of Pveg promoter | Pebbles lab |
| pSC-Pgrac-cre | Shuttle vector, in B. subtilis has ts DNA replication, encodes CRE recombinase under Pgrac promoter. Cm^{R} in Bacillus, Ap^{R} in E. coli | Present invention |
| pTSC | E. coli Ap^{R} Bacillus Em^{R} , CRE recombinase without promoter | BGSC (ECE204) (Yan et al., 2008) |
| p7Z6 | Ap^{R} | BGSC (ECE203) (Yan et al., 2008) |

TABLE 2

Bacillus strain doubling time.

| Bacillus | Doubling time (min) |
|---|---|
| wt168 | 6.3 ± 0.7 |
| Δ6 | 6.8 ± 1.9 |
| MM-W1 | 6.2 ± 1.2 |
| MM-W2 | 8.9 ± 0.8 |

### REFERENCES

Dunn, A. K. & J. Handelsman, (1999) A vector for promoter trapping in Bacillus cereus.Gene 226: 297-305.

Guohua YIN, E. S. L., Timothy S. TRAVERS, (2019) Systems, methods and composition of using rnase iii mutants to produce srna to control host pathogen infection. In

Koo, B. M., G. Kritikos, J. D. Farelli, H. Todor, K. Tong, H. Kimsey, I. Wapinski, M. Galardini, A. Cabal, J. M. Peters, A. B. Hachmann, D. Z. Rudner, K. N. Allen, A. Typas & C. A. Gross, (2017) Construction and Analysis of Two Genome-Scale Deletion Libraries for Bacillus subtilis. Cell Syst 4: 291-305 e297.

Phan, T. T. P., L. T. Tran, W. Schumann & H. D. Nguyen, (2015) Development of Pgrac100-based expression vectors allowing high protein production levels in Bacillus Subtilisname> and relatively low basal expression in Escherichia coli. Microbial Cell Factories 14: 72.

Sayre, R. T., Vinogradova-shah, Tatiana, (2020) Novel System for the Biocontrol of Pathogens in Aquaculture and Other Animal Systems. In. United States: Pebble Labs USA Inc. (Los Alamos, NM, US),

Yan, X., H. J. Yu, Q. Hong & S. P. Li, (2008) Cre/lox system and PCR-based genome engineering in Bacillus subtilis. Applied and Environmental Microbiology 74: 5556-5562.

1. A bacterial strain according to B. Subtilis sp. MM-W1 (ATCC patent deposit number PTA-126789) or B. Subtilis sp. MM-W2 (ATCC patent deposit number PTA-126790).

2. (canceled)

3. The bacterial strain of claim 1, wherein the bacterial strains are genetically engineered to express a heterologous nucleotide sequence, operably linked to a promoter, encoding a eukaryotic-like mRNA that may be introduced to and translated in a target host.

4. The bacterial strain of claim 1, wherein the bacterial strains are genetically engineered to express a heterologous nucleotide sequence, operably linked to a promoter, encoding an inhibitory RNA molecule configured to inhibit expression of at least one of the following:
at least one endogenous gene in a target host;
at least one gene of a target host pathogen; and
at least one gene involved in quorum sensing and/or biofilm formation; and.

5. The bacterial strain of claim 1, wherein the B. Subtilis sp. MM-W1 is genetically engineered to express a heterologous nucleotide sequence, operably linked to a promoter, encoding one or more of the following:
a heterologous double stranded RNA (dsRNA) molecule configured to inhibit expression of at least one target gene; and
a heterologous engineered RNaseIII configured for enhanced generation of small interfering RNAs (siRNAs) from catalytic cutting of said heterologous dsRNA molecule, wherein the siRNAs generated by the engineered RNaseIII are between 22-23 nucleotides in length.

6. The bacterial strain of claim 5, wherein said target gene comprises at least one of the following:
at least one endogenous gene in a target host;
at least one gene of a target host pathogen; and
at least one gene involved in quorum sensing and/or biofilm formation.

7. The bacterial strain of claim 1, wherein the bacterial strains are genetically engineered to express a heterologous truncated toxin peptide configured to competitively inhibit the activity of a target toxin produced by an EMS-causing bacterial pathogen.

8-9. (canceled)

10. The bacterial strain of claim 4, wherein said target host pathogen comprises a pathogen selected from the group comprising: white spot syndrome virus (WSSV), Early Mortality Syndrome (EMS) causing bacteria, a Vibrio bacterial pathogen, and quorum sensing (QS) in pathogenic bacteria.

11-14. (canceled)

15. The bacterial strain of claim 1, wherein the bacterial strains are genetically engineered to express a heterologous nucleotide sequence, operably linked to a promoter, encoding a heterologous quorum quenching molecule configured to remove exogenous AI-2 molecules from the environment and transport them into said bacterial strain.

16. The bacterial strain of claim 15, wherein said heterologous quorum quenching molecule comprises a heterologous lsr operon operably linked to a promotor configured to express at least one ATP-binding cassette transporter (ABC transporter) and at least one chaperone protein that actively pump exogenous autoinducer molecule AI-2 from the environment into said transformed bacterial cell.

17. (canceled)

18. The bacterial strain of claim 15, wherein said heterologous quorum quenching molecule comprises a homoserine lactonases (AHL lactonase) configured to inactivate exogenous AI-1 molecules.

19. (canceled)

20. The bacterial strain of claim 7, wherein said EMS-causing bacterial pathogen comprises a pathogenic species of Vibrio, wherein said target toxin produced by an EMS-causing bacterial pathogen comprises a Pir toxin comprising a PirA peptide and a PirB peptide, wherein said truncated toxin peptide configured to competitively inhibit the activity of a target toxin produced by an EMS-causing bacterial pathogen comprises a truncated PirB peptide, and a truncated PirA peptide.

21-28. (canceled)

29. The bacterial strain of claim 3, wherein said eukaryotic-like mRNA comprises a eukaryotic-like mRNA configured to generate a phenotypic change in said target host.

30. The bacterial strain of claim 29, wherein said phenotypic change comprises a phenotypic change selected from the group consisting of: increased growth; enhanced stress resistance; enhanced disease resistance; production of a naturally occurring compounds; production of a non-naturally occurring compounds; therapeutic pathogen bio-control; reduction in disease condition; a gene editing function.

31-34. (canceled)

35. A method of treating an aquatic animal or aquaculture environment, said method comprising administering a therapeutically effective amount of a bacterial strain according to claim 1 into an aquaculture environment or to an aquatic animal in need thereof.

36. The method of claim 35, wherein the method comprises treating infection in said aquatic animal, wherein said aquatic animal is, a shrimp in need thereof.

37. The method of claim 36, wherein said infection is by a pathogen selected from the group consisting of: white spot syndrome virus (WSSV), and an EMS causing bacteria.

38. The method of claim 35, wherein the aquatic animal is a shrimp, wherein the method comprises treating the aquatic animal to prevent biofilm formation by administering a therapeutically effective amount of the bacterial strain to a shrimp in need thereof.

39. The method of claim 35, wherein the method comprises treating the aquaculture environment to prevent biofilm formation, by administering a therapeutically effective amount of the bacterial strain to said aquaculture environment.

40. The bacterial strain of claim 3,wherein said nucleotide sequence is codon optimized for expression in Bacillus Subtilis.

41. A feed for aquatic animals comprising a bacterial strain according to claim 1.
